(19) United States
(12) Patent Application Publication
(10) Pub. No.: US 2015/0250826 A1
(43) Pub. Date: Sep. 10, 2015

(54) ISOLATION AND CHARACTERIZATION OF MUSCLE REGENERATING CELLS

(71) Applicant: Joslin Diabetes Center, Inc., Boston, MA (US)

(72) Inventors: Amy Wagers, Cambridge, MA (US); Alessandra Castiglioni, Brookline, MA (US); Simone Hettmer, Boston, MA (US)

(73) Assignee: Joslin Diabetes Center, Inc., Boston, MA (US)

(21) Appl. No.: 14/426,236

(22) PCT Filed: Sep. 5, 2013
(86) PCT No.: PCT/US2013/058227

§ 371 Date: Mar. 5, 2015

Related U.S. Application Data
(60) Provisional application No. 61/697,537, filed on Sep. 6, 2012.

(51) Int. Cl. A61K 35/34; G01N 33/569

(52) U.S. Cl. CPC A61K 35/34; G01N 33/56966; G01N 2333/70589; G01N 2333/70553; G01N 2333/70596; G01N 2333/70503; G01N 2333/70546

(57) ABSTRACT

Cell populations enriched for human myogenic progenitors are obtained by selection on the basis of expression of specific cell surface markers. The muscle progenitor cells are characterized as being CD45−, Mac-1−, GlycophorinA−, CD31− and CD34−, ITGA7hi and CD56 intermediate and methods of use thereof. Methods are provided for the separation and characterization of human myogenic cells, which are precursor cells having the ability to form muscle. The cells are identified and isolated from cells found within the pool of muscle satellite cells, located beneath the basal lamina of mature muscle fibers in the muscle tissue.

### BACKGROUND OF THE INVENTION

Stem cells have a capacity both for self-renewal and the generation of differentiated cell types. This multipotentiality makes stem cells unique. In addition to studying the important normal function of stem cells in the regeneration of tissues, researchers have further sought to exploit the potential of in situ and/or exogenous stem cells for the treatment of a variety of disorders. While early, embryonic stem cells have generated considerable interest, the stem cells resident in adult tissues may also provide an important source of regenerative capacity.

Somatic, or adult, stem cells are undifferentiated cells that reside in differentiated tissues, and have the properties of self-renewal and generation of differentiated cell types. These differentiated cell types may include all or some of the specialized cells in the tissue. For example, hematopoietic stem cells give rise to all hematopoietic lineages, but do not seem to give rise to stromal and other cells found in the bone marrow or other tissues. Sources of somatic stem cells include bone marrow, blood, the cornea and the retina of the eye, brain, skeletal muscle, dental pulp, liver, skin, and the lining of the gastrointestinal tract. Adult stem cells are usually quite sparse. Often they are difficult to identify, isolate, and purify. Often, somatic stem cells are quiescient until stimulated by appropriate growth signals.

Progenitor cells are similar to stem cells, but are usually considered to be distinct by virtue of lacking the capacity for self-renewal. Researchers often distinguish progenitor cells from stem cells in the following way: when a stem cell divides, one of the two new cells is often a stem cell capable of replicating itself again. Progenitor cells ultimately differentiate to produce mature daughter cells that replace cells that are damaged or dead, thus maintaining the integrity and functions of a tissue such as liver or brain.

Muscle tissue in adult vertebrates regenerates from reserve myogenic progenitor cells called satellite cells. Satellite cells are distributed throughout muscle tissue and are mitotically quiescent in the absence of injury or disease. Following recovery from damage due to injury or disease or in response to stimuli for growth or hypertrophy, satellite cells re-enter the cell cycle, proliferate and undergo differentiation into myoblasts, which fuse to form multinucleated myotubes and, new muscle fibers. The myoblasts ultimately yield replacement muscle fibers or fuse into existing muscle fibers, thereby increasing fiber number and/or fiber girth by the synthesis of contractile apparatus components. This process is illustrated, for example, by the nearly complete regeneration that occurs in mammals following induced muscle fiber degeneration or injury; the muscle progenitor cells proliferate and produce myoblasts that fuse together to regenerate muscle fibers.

Vertebrate muscles are thought to originate in the embryo from mesoderm-derived cells of the dorsal somites. During muscle development, some somite-derived myogenic progenitors do not differentiate into myofibers and instead are retained as muscle stem cells, or satellite cells, located beneath the basal lamina of muscle fibers. Satellite cells first appear in the limb muscles of mouse embryos between 16 and 18 days post conception (dpc). In neonatal mice, satellite cell nuclei comprise about 30% of myofiber-associated nuclei, but their number declines with age and only about 5% of myofiber nuclei in the muscles of adult mice represent satellite cells.

In injured adult muscle, satellite cell number and regenerative capacity remain nearly constant through multiple cycles of regeneration, suggesting that these cells may be capable of self-renewal, or that this population is maintained by self-renewing satellite cell precursors. Currently, satellite cells are defined both positionally, by their location beneath the basal lamina, and functionally, by their ability to undergo myogenic differentiation; however, potential heterogeneity in the function and/or origin of sublaminar myogenic cells may exist and has yet to be fully addressed.

In recent years, reports of adult skeletal muscle progenitors distinct from satellite cells have accumulated. For example, muscle-resident side population (muSP) cells, defined by their ability to exclude Hoechst 33342 and representing a population distinct from satellite cells, have been shown to contribute to myofibers when injected intramuscularly (McKinney-Freeman et al., 2002) or when co-cultured with myoblasts (Asakura, et al., (2002) J Cell Biol 159, 123-34), although muSP cells appear to lack myogenic activity when cultured alone.

Likewise, muscle-resident CD45⁺Sca-1⁺ cells fail to generate myogenic cells in vitro when cultured alone, but acquire myogenic potential when co-cultured with primary myoblasts or in response to muscle injury or activation of Wnt signaling by LiCl (Polesskaya, et al., (2003) Cell 113, 841-52).

In addition, cells with high proliferative potential and the ability to differentiate into multiple cell types, including muscle, neural, endothelial and hematopoietic lineages, have been isolated from muscle (Cao, et al., (2003) Nat Cell Biol 5, 640-6; Qu-Petersen et al. (2002) J Cell Biol 157, 851-64). Finally, bone marrow cells have been suggested by some researchers to contribute to myofibers when injected directly into injured muscle or intravenously into injured (Fukada, et al., (2002) J Cell Sci 115, 1285-93) or mdx dystrophic animals (Ferrari et al. (2001) Nature 411, 1014-5). Even single hematopoietic stem cells (HSC), which are capable of reconstituting the entire hematopoietic system (Wagers et al. (2002) Science 297, 2256-9), can contribute at a very low-level to skeletal myofibers following severe muscle injury (Camargo, et al., (2003) Nat Med 9, 1520-7; Corbel, et al., (2003) Nat Med 9, 1528-32).

Skeletal muscle accounts for up to 50% of human body mass and allows for locomotion by transmitting the contractile forces that move our bodies. Lifelong maintenance of skeletal muscle function relies on preserving its regenerative capacity, which involves a highly regulated process initiated by activation of normally quiescent muscle satellite cells (Wagers A J & Conboy I M (2005) Cellular and molecular signatures of muscle regeneration: current concepts and controversies in adult myogenesis. Cell 122(5):659-667). Satellite cells are located beneath the basal lamina of mature muscle fibers (Mauro A (1961) Satellite cell of skeletal muscle fibers. J Biophys Biochem Cytol 9:493-495) and express the canonical nuclear marker PAX7 (Reimann J, et al. (2004) Pax7 distribution in human skeletal muscle biopsies and myogenic tissue cultures. Cell Tissue Res 315(2):233-242; Seale P, et al. (2000) Pax7 is required for the specification of myogenic satellite cells. Cell 102(6):777-786). In mice, combinatorial analysis of cell surface markers allows direct discrimination and isolation by fluorescence-activated cell sorting (FACS) of highly regenerative muscle stem cells, called skeletal muscle precursor cells (SMPs), from the pool of myofiber-associated cells (U.S. Pat. No. 7,749,754 to Sherwood; Sherwood, et al., Cell, 119:543-554, 2004). In this previous work, adult mouse muscle cell progenitors that are capable of developing into skeletal muscle (i.e., myogenic; regenerating skeletal muscle) have been isolated from the greater pool of satellite cells found in the mouse muscle tissue (U.S. Pat. No. 7,749,754 to Sherwood; Sherwood, et al., Cell, 119:543-554, 2004). However, the identification of mouse satellite cell subpopulations that are capable of myogenic activity is of little help in the identification and isolation of similarly responding cells in humans. This is because the markers that identify the suitable mouse cell population(s) have little to no relation to the markers that may identify a similarly acting human cell population(s). The difference between species in the identification of suitable distinguishing cell markers is attributed to, for example, genetic differences, differences in the host's cellular microenvironment and/or host physiological differences. Thus, as is well known in the art, the identification of markers for one species is of little if any help in the identification of cell markers for a similar cell population of a differing species.

Thus, what is needed are compositions and methods for identifying human muscle cell populations that are suitable for the regeneration of muscle tissue (i.e., have myogenic ability) as well as the identification and isolation of those muscle cell populations. In doing so, the researcher and clinician will have the ability to manipulate muscle regeneration. Further, satellite cells have been shown to play a role in muscle tumor generation and growth (Hettmer, et al., PNAS, 2011, 108(50):20002-20007). Thus, the identification of and isolation of those muscle cell populations may be instrumental in the study of sarcomas and thereby aid in the developing treatment options. Therefore, characterization of human stem and progenitor cells having myogenic potential is of great need and interest.

### SUMMARY OF THE INVENTION

Methods are provided for the separation and characterization of human myogenic cells, which are precursor cells having the ability to form muscle. The cells are identified and isolated from cells found within the pool of muscle satellite cells, located beneath the basal lamina of mature muscle fibers in the muscle tissue. The ability to form muscle may be evidenced by various indicia, including expression of myogenic proteins; autonomous in vitro myogenic colony-forming capacity; myogenic capacity in co-culture with isolated muscle-resident myogenic cells; in vivo contribution to myofibers in injured muscle; and engraftment of the myofiber-associated compartment in vivo following intramuscular injection and subsequent maintenance of myogenic-colony forming capacity. Populations enriched for myogenic progenitors may be obtained by selection on the basis of expression of specific cell surface markers. These human muscle progenitor cells have been identified and isolated for the first time in the present invention and are characterized as being CD45⁻MAC1⁻GlycophorinA⁻CD34⁻, and may further be characterized as CD45⁻MAC1⁻GlycophorinA⁻CD31⁻CD34⁻ with, optionally ITGA7^{hi}CD56^{intermediate} expression.

The identified and isolated human muscle precursor cells (non-modified and/or genetically modified) are useful in autologous and allogeneic transplantation, particularly for the regeneration of skeletal muscle, e.g., in the treatment of muscle disorders such as muscular dystrophies, myopathies, chanelopathies; following traumatic damage; and the like. The cells are also useful for experimental evaluation, and as a source of lineage and cell specific products, including mRNA species useful in identifying genes specifically expressed in these cells, and as targets for the discovery of factors or molecules that can affect them. Further, the cells (when modified) are useful for model systems wherein sarcomas (e.g., rhabdomyosarcoma) are induced that can be used to, for example, test treatments and screen for agents that are beneficial in the treatment of sarcomas and similar cancers.

Further, in vitro and in vivo systems are provided for the growth and analysis, including clonal analysis, of myogenic cells. Clonogenic assays may be performed in vitro in the presence or absence of additional co-cultured myofiber associated cells, where different cell populations vary in their ability to generate myogenic colonies.

### BRIEF DESCRIPTION OF THE DRAWINGS

FIG. 1 shows the isolation of distinct subsets of myogenic and non-myogenic cells from human muscle by flow cytometry. 1A (left and right panels) & 1B show FACS histograms displaying the isolation of CD45⁻MAC1⁻Gycophorin⁻CD34⁻ITGA7^{hi}CD56^{intermediate} cells. Starting cells were MFA cells isolated from human adult or fetal muscle tissue. FIG. 1A, left panel, shows a gating on cells negative for a cocktail of antibodies recognizing CD45, MAC1, CD31 and Glycophorin. FIG. 1A, right panel, shows gatings on CD34 positive and CD34 negative cells of the CD45, MAC1, CD31 and Glycophorin negative cells from FIG. 1A, left panel. FIG. 1B shows three gatings on CD56 and ITGA7 expression of the CD34 negative subpopulation of cells of FIG. 1A, right panel.

FIG. 2: Isolation of distinct subsets of myofiber-associated (MFA) cells from human skeletal muscle. (A) Representative FACS plots showing progressive gating of subsets of human MFA cells, which can be discriminated by combinatorial staining for CD45, MAC1, Glycophorin and CD34. (B) CD45⁻MAC1⁻Gycophorin⁻CD34⁻ cells (lower gate in right hand histogram in (A)) are capable of efficient myogenic differentiation into Desmin-positive (light grey: shown as long, stained fibrils; darker speckling is Hoechst staining) multi-nucleated myofibers (B, left panels), while CD45⁻MAC1⁻Glycophorin⁻CD34⁺ cells (upper gate in right hand histogram in (A)) lack myogenic activity (Hoechst staining only) and adopt a fibroblastic morphology in vitro (right panels).

FIG. 3 shows in vivo engraftment of hSMPs in mouse skeletal muscle. Engrafted hMFA cells were detected in transplanted NSG mouse muscle by co-staining for human species-specific h-Spectrin (red), Laminin (green) and Dapi (blue). (a-b) Species-specific staining for h-Spectrin is strongly positive in human muscle (a) and absent in mouse muscle (b). (c) Unfractionated hMFA cells engrafted to form h-Spectrin positive cells in 4 out of 4 transplanted mice. (d) Sorted hSMP cells engrafted to form h-Spectrin positive cells in 6 out of 22 transplanted mice (left upper panel). h-Spectrin positive, engrafted cells aligned with Laminin-positive myofibers in muscles transplanted with hSMPs (upper panels) and hMFA cells (lower panels), thereby demonstrating that engrafted cells contribute to the architecture of recipient muscles. Representative images at 40× magnification.

FIG. 4 shows the distinct transcriptional signatures of hSMP and CD34⁺ cells. Principal Component Analysis (a) and hierarchical clustering (b) demonstrate distinct gene expression signatures of CD34+ cells (blue, CD34⁺), hSMPs (red, CD34⁻ITGA7⁺CD56^{int}) and parental hMFA cells (green), with no overlap between the group of genes that are differentially regulated (up or down; >1.5-fold difference and p<0.05) in hSMPs vs hMFAs and hFAPs vs hMFAs, respectively (c). Microarray analysis was performed using 3-4 (see (b)) biologically independent, freshly sorted hSMP (CD34⁻CD56^{int}ITGA7^{hi}), CD34⁺ and unfractionated hMFA cell samples. (d) Microarray analyses demonstrated increased expression of muscle-lineage genes (PAX7, MYF5, CDH15, MYOD, MYOG) in hSMPs and increased levels of adipocyte-lineage genes (PPARG, FABP4, and COL1A1) in CD34⁺ cells. Osteolineage genes (ALPL, BGLAP and RUNX2) are present in both hSMPs and CD34⁺ cells. (e) Expression of PAX7, MYF5, PPARG, FABP4, BGLAP and RUNX2 (relative to GAPDH) was evaluated by qPCR in hSMPs compared to CD34⁺ cells obtained from 2 biologically independent fetal hSMP (CD34⁻CD56^{int}ITGA7^{hi}) and 3 biologically independent CD34⁺ cells samples. Levels of PAX7 and MYF5 are 512-670 fold greater, and expression of PPARG, FABP4, BGLAP and RUNX2 is 8-60-fold lower in hSMPs cells as compared to CD34⁺ cells.

FIG. 5 shows adult CD34⁻CD56^{int}ITGA7^{hi} hMFA cells are PAX7-expressing myogenic precursor cells. (a) FACS gating strategy for isolation of CD45⁻CD11b⁻GlyA⁻CD31⁻CD34⁻CD56^{int}ITGA7^{hi} cells within the pool of 7AAD⁻Ca⁺ adult hMFA cells. (b) Enrichment of PAX7 expression in adult CD34⁻CD56^{int}ITGA7^{hi} hMFA cells, assessed by IF. 89±7% (mean±s.d) of adult CD34⁻CD56^{int}ITGA7^{hi} hMFA cells express PAX7 (as indicated in the middle panels—red in original). Nuclei were marked by Dapi stain (as indicated in the left hand panels—blue in original). The right hand panels combine the Dapi and PAX7 staining. (c) Myogenic differentiation assays using human adult MFA cell subsets show that CD34 expression discriminates between myogenic and non-myogenic adult hMFA cells, and myogenic activity is highly enriched in adult CD34⁻CD56^{int}ITGA7^{hi} cells. (d) Numbers of hMFA or (e) hSMP cells per gram of tissue, or (f) frequency of hSMP cells, were compared for adult or fetal muscle. Statistical significance was evaluated by unpaired, two-tailed t-test. Representative images were taken at 20× magnification.

FIG. 6 shows osteogenic differentiation capacity of adult hMFA subsets. CD34⁻CD56^{int}ITGA7^{hi} adult hSMP cells, obtained from three different adult donors (BI111212, BI111912, BI110512), formed Alizarin Red positive calcium deposits. Thus, osteogenic differentiation capacity of hSMPs is retained in adult skeletal muscle. Images were taken at 4× magnification.

FIG. 7: Kras, p16p19^{null} mouse sarcoma model. (A) Experimental design. Mouse myogenic precursor cells and Sca1⁺ cells were freshly isolated by FACS from the myofiber-associated cell compartment of p16p19^{null} mice, infected with Kras(G12V)-pGIPZ-IRES-GFP lentivirus, and injected into the gastrocnemius muscles of NOD.SCID recipient mice. Recipient muscles were preinjured by cardiotoxin injection to enhance engraftment (Cerletti, et al., Cell, 2008, 134(1):37). (B) Both satellite cells (lower line) and Sca1⁺ cells (upper line) rapidly induced tumors in the majority of mice. (C) Satellite cells gave rise to pleomorphic rhabdomyosarcomas expressing Myogenin (dark stain shown in C), as well as MyoD and Desmin (not shown). Sca1+ cells induced sarcomas lacking these myogenic markers (C and data not shown).

FIG. 8: In vitro screening of candidate targets. Sarcoma cells were exposed to increasing concentrations of Torin (10, 50, 250 nMol), Rapamycin (10, 50, 100 nMol), RL0061425 (10, 50, 100, 500, 1000 nMol), SB525334 (1, 10, 50, 100, 500 nMol), SD208 (10, 50, 100, 500, 1000 nMol), and Ethanol and DMSO (as a control). Cell growth was evaluated by determining the fold-increase in MTT-uptake over a defined time period (48 hours for the fast-growing mouse Kras; p16p19^{null} rhabdomyosarcoma cell line and 96 hours for the slower-growing mouse Kras; p16p19^{null} sarcoma line and the human rhabdomyosarcoma cell line RD. The human rhabdomyosarcoma cell line RD is known to those of skill in the art. mTOR inhibitors (shown are: Torin at 250 nMol and Rapamycin at 100 nMol concentration marked in medium grey in all three panels, A-C) reduced the growth of all three cell lines. The TGF inhibitor SD208 inhibited the growth of the mouse sarcoma cell lines at ≧500 nMol (marked in light grey in panels A, B), while the TGF inhibitors RL0061425 and SB525334 had no major effect.

FIG. 9: Lentiviral (LV) delivery of shRNAs to muscle progenitor cells and sarcoma cells. (A-C) Sorted satellite cells (see FIG. 1) were transduced with LV encoding GFP only (pSicoR-GFP, B) or GFP plus short hairpin RNA (shRNA) targeting the transcription factor Egr1 (pSicoRshEGR1, C). Figure shows fluorescence images of non-transduced (A) and LV-infected (B, C) cells. shEGR1-transduced cells showed reduced proliferation and enhanced differentiation (not shown). (D) Human RD cells were transduced with puromycin-selectable pLKO lentiviruses carrying shRNA against STK33 (2 hairpins tested, 78 &79) or S6K (2 hairpins tested, 58 & 59), two kinases implicated by our studies in sarcoma growth. Puromycin was added 1 day after infection, to select for transduced cells, and proliferation was measured by MTS assay after 2 days (first bar of each set), 4 days (2^{nd} bar) or 6 days (last bar). Data are displayed as relative growth compared to day 2. RD cells transduced with STK33 or S6K shRNAs grow less than cells in control cultures.

### DESCRIPTION OF THE SPECIFIC EMBODIMENTS

Methods are provided for the separation and characterization of human myogenic progenitor cells; and compositions of cells enriched for human myogenic progenitors are provided. The present Inventors developed a procedure for the identification and isolation of adult and fetal human progenitor cells. The identified cells are useful for transplantation into the muscles of persons having injured or diseased muscles and useful for the modeling of sarcomas. Further, the cells and model systems based on the cells are useful for the screening of therapeutic agents suitable for the inhibition or treatment of sarcomas or the stimulation of muscle growth and repair. Use of a procedure that separates muscle-resident cells into a myofiber-associated compartment highly enriched for satellite cells, and a separate interstitial cell preparation, has allowed direct analysis of the myogenic potential of these cells. The ability to sort these distinct populations freshly from human muscle cell lineage relationships in the differentiation of muscle stem cells and progenitors as well as the determination of the signaling pathways and gene expression dynamics important for maintaining muscle-resident cell populations.

It is shown herein that the identified and isolated muscle cells are able to fulfill various criteria for a myogenic progenitor, including the ability to generate autonomous in vitro myogenic colonies. Further, the isolated muscle cells will contribute in vivo to myofibers in injured muscle and engraftment of the myofiber-associated compartment in vivo following intramuscular injection and subsequent maintenance of myogenic-colony forming capacity.

The subject cells of the present invention are useful for transplantation, particularly for the regeneration of skeletal muscle, e.g., in the treatment of muscle disorders such as muscular dystrophies, myopathies, chanelopathies; following traumatic damage; and the like. The cells may also be used for experimental evaluation, and as a source of lineage and cell specific products, including mRNA species useful in identifying genes specifically expressed in these cells, and as targets for the discovery of factors or molecules that can affect them.

The endogenous myofiber associated cells having muscle precursor cell activity were found to be CD45⁻MAC1⁻GlycophorinA⁻CD34⁻ and CD31⁻, and may further be characterized as CD45⁻MAC1⁻GlycophorinA⁻CD31⁻CD34⁻ITGA7^{hi}CD56^{intermediate}.

In one embodiment of the present invention, a method of enrichment is contemplated for a composition comprising a population of human myogenic progenitor cells, wherein at least 80% of the cells in said population are myofiber associated, CD45−, Mac-1−, GlycophorinA−, CD31−, CD34−, ITGA7+ and CD56+, the method comprising: dissociating human muscle tissue to provide a population of myofiber associated cells; combining reagents that specifically distinguish CD45−, Mac1−, GlycophorinA−, CD31− and CD34− respectively, within said population of myofiber associated cells; and selecting for those cells that are CD45−, Mac1−, GlycophorinA−, CD31−, CD34−, ITGA7+ and CD56+; wherein said selected population of cells are capable of forming myogenic colonies. The method may further comprise selecting for cells that are selected for ITGA7^{hi} and CD56^{intermediate}.

In another embodiment of the present invention a composition is contemplated comprising a population of isolated human myogenic progenitor cells, said cells isolated from human muscle tissue and selected for CD45−, Mac1−, GlycophorinA−, CD31− and CD34−, ITGA7+ and CD56+ expression. The composition may further comprise cells that are selected for ITGA7^{hi} and CD56^{intermediate} expression.

In another embodiment of the present invention a method of regenerating muscle tissue in a patient is contemplated, comprising: providing; a composition comprising a population of human myogenic progenitor cells, said cells isolated from human muscle tissue and selected for CD45−, Mac1−, GlycophorinA−, CD31− and CD34−, ITGA7+ and CD56+ expression and a patient in need of muscle tissue regeneration; and introducing said composition into said patient in the location where said muscle regeneration is needed, thereby promoting the regeneration of muscle tissue. The method may further comprise cells that are selected for ITGA7^{hi} and CD56^{intermediate} expression.

In another embodiment of the present invention a method is contemplated for screening for pharmacological agents effective for promoting muscle cell growth, said method comprising: providing: a composition comprising a population of human myogenic progenitor cells, said cells isolated from human muscle tissue and selected for CD45−, Mac1−, GlycophorinA−, CD31− and CD34−, ITGA7+ and CD56+ expression and a pharmacological agent; contacting said cells with said pharmacological agent to create treated cells; and determining the rate of cell growth as compared to an essentially identical population of control cells that were not contacted with said pharmacological agent, wherein an increased cell growth rate of the treated cells as compared to the control cells is indicative of promotion of cell growth by the pharmacological agent. The method may further comprise using cells that are selected for ITGA7^{hi} and CD56^{intermediate} expression. Optionally, the method may also include determining the efficiency of myogenic differentiation as compared to an essentially identical population of control cells that were not contacted with said pharmacological agent, wherein an increased myogenic differentiation rate of the treated cells as compared to the control cells is indicative of promotion of muscle cell differentiation by the pharmacological agent.

In another embodiment of the present invention a method is contemplated of modeling human soft tissue sarcoma, comprising: providing; a composition comprising a population of human myogenic progenitor cells, said cells isolated from human muscle tissue and selected for CD45−, Mac1−, GlycophorinA−, CD31− and CD34−, ITGA7+ and CD56+ expression and an immune deficient mouse; modifying the isolated human myogenic progenitor cells by introducing into said cells genetic mutations suitable for activating mutations in Ras proteins and suitable for the disruption of [cell cycle regulation] cyclin-dependent kinase inhibitor 2A (CDKN2A) to create modified cells; and introducing into and growing said modified cells in said immune deficient mouse. The method may further comprise using cells that are selected for ITGA7^{hi} and CD56^{intermediate} expression. It is contemplated that other cell cycle regulators and cyclin-dependent kinase inhibitors will be effective in the present invention.

The cells identified and isolated by the present invention are also useful for reconstitution or regeneration of muscle function in a recipient. Allogeneic cells (i.e., immune compatible cells from donors) may be used for progenitor cell isolation and subsequent transplantation. For example, where the disease conditions result from genetic defects in muscle cell function such as is the case with muscular dystrophies. Where the muscle dysfunction arises from conditions such as trauma, the subject cells can be isolated from autologous tissue (i.e., the recipient's own, undamaged muscle), and used to regenerate function. Autologous cells can also be genetically modified, in order to correct disease conditions results from genetic defects.

It is to be understood that this invention is not limited to the particular methodology, protocols, cell lines, and reagents described, as such may vary. It is also to be understood that the terminology used herein is for the purpose of describing particular embodiments only and is not intended to limit the scope of the present invention, which will be limited only by the appended claims.

As used herein the singular forms “a,” “an,” and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Myogenic Progenitors. As used herein, the term “myogenic progenitor” is used to refer to cells that can form muscle. For many purposes, the primary requirement is an ability to contribute to myofiber formation in vivo, e.g., in injured muscle.

Additional criteria for myogenicity include the expression of myogenic proteins, which include the intermediate filament protein desmin, myogenic transcription factors MyoD, Myf-5 and Pax-7.

Under myogenic conditions in vitro, myogenic progenitor cells will generally autonomously give rise to myogenic colonies. Myogenic conditions may include the presence of a substrate, such as collagen or laminin, where medium may include βFGF, IGF or other growth factors. The growth conditions may be changed to fusion conditions by reduction of growth factors for myofiber formation.

The stem/progenitor capability of myogenic progenitors may be evidenced by the ability to engraft and repopulate the myofiber-associated compartment in vivo following intramuscular injection, and subsequent maintenance of myogenic-colony forming capacity.

The term “muscle cell” as used herein refers to any cell that contributes to muscle tissue. Myoblasts, satellite cells, myotubes and myofibers are all included in the term “muscle cells.” Muscle cell effects may be induced within skeletal, cardiac and smooth muscles, particularly with skeletal muscle. Other cell types may be found in muscle tissue such as nerve cells, cells constituting blood vessels, blood cells, etc., but are not muscle cells.

The activation of satellite cells in muscle tissue can result in the production of new muscle cells in the patient. Muscle regeneration as used herein refers to the process by which new muscle fibers form from muscle progenitor cells. A therapeutic composition will usually confer an increase in the number of new fibers by at least 1%, more preferably by at least 20%, and most preferably by at least 50%. A therapeutic composition of the identified and isolated human myogenic progenitor cells of the present invention can be used in a therapeutic capacity. Muscle growth is measured by, for example, the increase in the fiber size and/or by increasing the number of fibers. Muscle growth may also be measured by an increase in wet weight, an increase in protein content, an increase in the number of muscle fibers, an increase in muscle fiber diameter, etc. An increase in growth of a muscle fiber can also be defined as an increase in the diameter where the diameter is defined as the minor axis of ellipsis of the cross section.

Muscle regeneration can also be monitored by the mitotic index of muscle, that is, the fraction of cells in the culture which have labeled nuclei when grown in the presence of a tracer which only incorporates during S phase (i.e., BrdU) and the doubling time is defined as the average S time required for the number of cells in the culture to increase by a factor of two. For example, cells may be exposed to a labeling agent for a time equivalent to two doubling times. Productive muscle regeneration may be also monitored by an increase in muscle strength and agility although this may also signal increase in size (i.e., girth) or strength of preexisting fibers.

Muscle regeneration may also be measured by quantitation of myogenesis, i.e., fusion of myoblasts to yield myotubes. An effect on myogenesis results in an increase in the fusion of myoblasts and the enablement of the muscle differentiation program. For example, myogenesis may be measured by the fraction of nuclei present in multinucleated cells relative to the total number of nuclei present. Myogenesis may also be determined by assaying the number of nuclei per area in myotubes or by measurement of the levels of muscle specific proteins by Western analysis.

Muscle fiber survival often refers to the prevention of loss of muscle fibers as evidenced by necrosis or apoptosis or the prevention of other mechanisms of muscle fiber loss. Muscles can be lost from injury, atrophy, and the like, where atrophy of muscle refers to a significant loss in muscle fiber girth. When muscle fiber replacement equals muscle fibers loss the result is a net balance with no increase or decrease in muscle fiber, although the energy expended by the subject may reveal itself in other physiological manners.

Positive and negative staining. The subject myogenic progenitor cells are characterized by their expression of cell surface markers. While it is commonplace in the art to refer to cells as “positive” or “negative” for a particular marker, actual expression levels are a quantitative trait. The number of molecules on the cell surface can vary by several logs, yet still be characterized as “positive.” Thus, cells identified as “low,” “hi” or “intermediate” may also be identified as “positive.” It is also understood by those of skill in the art that a cell which is negative for staining, i.e., the level of binding of a marker specific reagent is not detectably different from a control, e.g., an isotype matched control; may express minor amounts of the marker. Sometime, cells that are identified as “low” may be considered “negative” or, at least, not “positive” depending on the staining levels of other markers or cells in the sample or experiment. These characterizations of the level of staining permit subtle distinctions between cell populations.

The staining intensity of cells can be monitored by flow cytometry, where lasers detect the quantitative levels of fluorochrome (which is proportional to the amount of cell surface marker bound by specific reagents, e.g., fluorochrome labeled antibodies). Flow cytometry, or FACS (fluorescence activated cell sorter), can also be used to separate cell populations based on the intensity of binding to a specific reagent, as well as other parameters such as cell size and light scatter. Although the absolute level of staining may differ with a particular fluorochrome and reagent preparation, the data can be normalized to a control. The use of flow cytometry is well known by those of ordinary skill in the art.

As is known to one of ordinary skill in the art, in order to normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than minimally stained or unstained cells. When displayed in this manner, it is clear that the cells falling in the highest log of staining intensity are bright, while those in the lowest intensity are negative. The “low” positively stained cells have a level of staining above the brightness of an isotype matched control, but it is not as intense as the most brightly staining cells normally found in the population. Low positive cells may have unique properties that differ from the negative and brightly stained positive cells of the sample. An alternative control may utilize a substrate having a defined density of marker on its surface, for example a fabricated bead or cell line, which provides the positive control for intensity.

Sources of Progenitor Cells. Ex vivo and in vitro cell populations useful as a source of cells may include fresh or frozen muscle fiber cell populations, usually skeletal muscle, obtained from embryonic, fetal, pediatric or adult tissue. Frozen tissue sources have usually been preserved such that the cells do not rupture upon the freeze/thaw of the cell or tissue sample. Preservation of cells in suitable levels of DMSO, for example, is a typical method of limiting the formation of ice crystals in the cells sample to limit cell damage and destruction. The methods can include further enrichment or purification procedures or steps for cell isolation by positive selection for other cell specific markers. The progenitor cells may be obtained from any mammalian species, e.g., human, equine, bovine, porcine, canine, feline, rodent, e.g., mice, rats, hamster, primate, etc., although the markers selective of a particular target cell type is highly variable between species, and markers that identify progenitor cells in one species do not predict markers that will identify the analogous cells in a different species. In this regard, the knowledge of identifying markers of myogenic progenitors of one species is of little use in determining what markers or marker combinations may be useful in identifying and isolating suitable myogenic progenitor cells in another species.

Markers. The markers for selection of myogenic progenitors will vary with the specific cells and specific species. As described above, a number of well-known markers can be used for positive selection and negative selection.

With regard to the present invention, markers and marker combinations of interest include, for example and without limitation, positive selection for CD31, ITGA7 and CD56 and negative selection for CD45, CD34, Mac-1, GlycophorinA CD31 and CD11b.

Specific Binding Member. The term “specific binding member” or “binding member” as used herein refers to a member of a specific binding pair, i.e., two molecules, usually two different molecules, where one of the molecules (i.e., first specific binding member) through chemical or physical means specifically binds to the other molecule (i.e., second specific binding member). The complementary members of a specific binding pair are sometimes referred to as a ligand and receptor; or receptor and counter-receptor. Such specific binding members are useful in positive and negative selection methods. Specific binding pairs of interest include carbohydrates and lectins; complementary nucleotide sequences; peptide ligands and receptor; effector and receptor molecules; hormones and hormone binding protein; enzyme cofactors and enzymes; enzyme inhibitors and enzymes; antibodies and antigens; etc. The specific binding pairs may include analogs, derivatives and fragments of the original specific binding member. For example, a receptor and ligand pair may include peptide fragments, chemically synthesized peptidomimetics, labeled protein, derivatized protein, etc. Further, a receptor may have multiple ligands and a ligand may bind more than one receptor with actual binding depending on the immediate, local physiological and environmental conditions. Further still, binding of receptors and ligands may show varying levels of sensitivity and specificity depending on the immediate, local physiological and environmental conditions.

Especially useful reagents are antibodies specific for markers present on the desired cells (for positive selection) and undesired cells (for negative selection). Whole antibodies may be used, or fragments, e.g., Fab, F(ab′)₂, light or heavy chain fragments, etc. Such selection antibodies may be polyclonal or monoclonal and are generally commercially available or alternatively, readily produced by techniques known to those skilled in the art. Antibodies selected for use will have a low level of non-specific staining and will usually have an affinity of at least about 100 μM for the antigen.

Antibodies for selection may be coupled to a label. Labels include magnetic beads, which allow for direct separation, biotin, which can be removed with avidin or streptavidin bound to a support, fluorochromes, which can be used for flow cytometry with a fluorescence activated cell sorter (FACS), or the like, to allow for ease of separation of the particular cell type. Fluorochromes that find use include phycobiliproteins, e.g., phycoerythrin and allophycocyanins, fluorescein and Texas red, cy7, cy5. Frequently each antibody is labeled with a different fluorochrome, to permit independent sorting for each marker. In other work multiple antibodies may be coupled to the same fluorochrome to aid in the efficient removal or selection of cells based on multiple characteristics. Likewise, one or more antibodies may be coupled to magnetic beads, by techniques known to those of ordinary skill in the art, to allow the removal or selection of cells based on a single or multiple characteristics. The exact method for coupling to a label is not critical to the practice of the invention, and a number of alternatives are known in the art. Direct coupling attaches the antibodies to the label. Indirect coupling can be accomplished by several methods. The antibodies may be coupled to one member of a high affinity binding system, e.g. biotin, and the particles attached to the other member, e.g., avidin. One may also use second stage antibodies that recognize species-specific epitopes of the antibodies, e.g., anti-mouse Ig, anti-rat Ig, etc. Indirect coupling methods allow the use of a single labeled entity, e.g., antibody, avidin, etc., with a variety of separation antibodies. Multiples of the second stage antibodies may be coupled to the same fluorochrome to aid in the efficient removal or selection of cells based on multiple characteristics.

Enrichment Methods

The subject myogenic cells are separated from a complex mixture of cells by techniques that enrich for cells having the characteristics as described. For example, a muscle sample may initially be prepared by dissociation of myofibers. From this population, cells may be selected for size, viability and for expression of one or more cell surface markers.

In one embodiment the present invention provides for the enrichment (selection) of muscle cells by the methods discussed herein and by techniques known in the art that are CD45−, Mac1−, GlycophorinA−, CD31−, CD34−, ITGA7+ and CD56+; especially wherein said selected population of cells are capable of forming myogenic colonies. The enrichment methods may further comprise selecting for cells that are selected for ITGA7^{hi} and CD56^{intermediate}.

Dissociation of muscle usually includes digestion with a suitable protease, e.g., collagenase, dispase, trypsin, etc., followed by trituration until dissociated into myofiber fragments. Fragments are then washed and further enzymatically dissociated to generate a population of myofiber associated cells. An appropriate solution is used for dispersion or suspension. Such solution will generally be a balanced salt solution, e.g., normal saline, PBS, Hanks balanced salt solution, etc., or suitable cell culture media, supplemented with fetal calf serum or other naturally occurring factors, in conjunction with an acceptable buffer at low concentration, generally from 5-25 mM. Convenient buffers include HEPES, phosphate buffers, lactate buffers, etc.

Separation of the subject cell population will then use affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., plate, or other convenient technique. Techniques providing accurate separation include flow cytometry using fluorescence activated cell sorters (FACS), which can have varying degrees of sophistication, such as multiple color channels, low angle and obtuse light scattering detecting channels, impedance channels, etc. The cells may be selected against dead cells by employing dyes associated with dead cells (propidium iodide (PI), 7-AAD) or dyes associated with viable cells (e.g., calcein). Any technique may be employed which is not unduly detrimental to the viability of the selected cells.

The affinity reagents may be specific receptors or ligands for the cell surface molecules indicated above. The details of the preparation of antibodies and their suitability for use as specific binding members are well known to those skilled in the art. Of particular interest is the use of antibodies as affinity reagents.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium which maintains the viability of the cells. A preferred medium is phosphate buffered saline containing from 0.1 to 0.5% BSA. Various media are commercially available and may be used according to the nature of the cells, including Dulbeccos Modified Eagle Medium (dMEM), Hank's Basic Salt Solution (HBSS), Dulbeccos phosphate buffered saline (dPBS), RPMI, Iscoves medium, PBS with 5 mM EDTA, etc., frequently supplemented with fetal calf serum, BSA, HSA, Ham's F10, Ham's F-12, etc.

The labeled cells are then separated as to the phenotype described above. The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscoves medium, etc., frequently supplemented with fetal calf serum (or other serum as suitable).

Compositions highly enriched for muscle engrafting activity are achieved in this manner. The subject population will be at or about 50% or more of the cell composition, 75% or more of the cell composition, 80% or more of the cell composition and usually at or about 90% or more of the cell composition, and may be as much as about 95% or more of the live cell population. The enriched cell population may be used immediately, or may be frozen at liquid nitrogen temperatures and stored for long periods of time, being thawed and capable of being reused. The cells will usually, for example, be stored in 10% DMSO, 90% FCS medium although other combinations are known to or can be identified by those of skill in the art. Once thawed, the cells may be expanded by use of growth factors and/or feeder cells for proliferation and differentiation, as is known by one of ordinary skill in the art.

The compositions thus identified, isolated and obtained by the present invention have a variety of uses in clinical therapy, research, development, and commercial purposes. For therapeutic purposes, for example, myocytes and their precursors may be administered to enhance tissue maintenance or repair of muscle for any perceived need, such as an inborn error in metabolic function, the effect of a disease condition or the result of significant trauma.

To determine the suitability of cell compositions for therapeutic administration, the cells can first be tested in a suitable animal model. At one level, cells are assessed for their ability to survive and generate the desired phenotype in vivo. Cell compositions are preferably administered to immunodeficient animals (such as nude mice, or animals rendered immunodeficient chemically or by irradiation). Tissues are harvested after a period of regrowth, and assessed as to whether the administered cells or progeny thereof are still present.

Identification of transplanted cells and their progeny can be performed by administering cells that express a detectable label (such as green fluorescent protein, or β-galactosidase); that have been prelabeled (for example, with BrdU or [³H] thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

Where the differentiating cells are cells of the myocyte lineage, suitability can also be determined in an animal model by assessing the degree of muscle regeneration that ensues from treatment with the differentiating cells of the invention. A number of animal models are known by those of skill in the art and are available for such testing. For example, muscle can be injured as described in the Examples. Injured sites are treated with cell preparations of this invention, and the muscle tissue is examined by histology for the presence of the cells in the damaged area.

Cells may be genetically altered in order to introduce genes useful in the differentiated cell, e.g., repair of a genetic defect in an individual, selectable marker, etc. Cells may be genetically altered by transfection or transduction with a suitable vector, homologous recombination, or other appropriate technique, so that they express a gene of interest. In one embodiment, cells are transfected with genes encoding dystrophin. In other embodiments, a selectable marker is introduced, to provide for greater purity of the desired cell. Cells may be genetically altered using vector containing supernatants over a 8-16 h period, and then exchanged into growth medium for 1-2 days. Genetically altered cells are selected using a drug selection agent such as puromycin, G418, or blasticidin, and then recultured. Genetically modified cells can also be selected for a detectable marker, e.g., GFP, etc., by cell sorting methods known in the art.

Many vectors useful for transferring exogenous genes into target mammalian cells are available. The vectors may be episomal, e.g., plasmids, virus derived vectors such cytomegalovirus, adenovirus, etc., or may be integrated into the target cell genome, through homologous recombination or random integration, e.g., retrovirus derived vectors such MMLV, HIV-1, ALV, etc., or by lentiviral vectors such as pGIPZ, pLKO, etc. Retrovirus based vectors have been shown to be particularly useful when the target cells are progenitor cells. For example, see Schwarzenberger, et al., (1996) Blood 87:472-478; Nolta, et al., (1996) P. N. A. S. 93:2414-2419; and Maze, et al., (1996) P. N. A. S. 93:206-210.

Combinations of retroviruses and an appropriate packaging line may be used, where the capsid proteins will be functional for infecting the target cells. Usually, the cells and virus will be incubated for at least about 24 hours in the culture medium. The cells are then allowed to grow in the culture medium for short intervals in some applications, e.g., 24-73 hours, or for at least two weeks, and may be allowed to grow for five weeks or more, before analysis. Commonly used retroviral vectors are “defective,” i.e., unable to produce viral proteins required for productive infection. Replication of the vector requires growth in the packaging cell line.

Lentiviral vectors such as those based on HIV or FIV gag sequences can be used to transfect non-dividing cells, such as the resting phase of human stem cells (see. e.g., Uchida, et al., (1998) PNAS. 95(20):11939-44).

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g., MMLV, are capable of infecting most mouse and rat cell types. Ecotropic packaging cell lines include BOSC23 (Pear, et al., (1993) P. N A S. 90:8392-8396). Retroviruses bearing amphotropic envelope protein, e.g. 4070A (Danos, et al., supra.), are capable of infecting most mammalian cell types, including human, dog and mouse. Amphotropic packaging cell lines include PA12 (Miller, et al, (1985) Mol. Cell. Biol. 5:431-437); PA317 (Miller, et al., (1986) Mol. Cell. Biol. 6:2895-2902) GRIP (Danos, et al., (1988) PNAS 85:6460-6464). Retroviruses packaged with xenotropic envelope protein, e.g., AKR env, are capable of infecting most mammalian cell types, except murine cells.

The vectors may include genes that must later be removed, e.g., using a recombinase system such as Cre/Lox, or the cells that express them destroyed, e.g., by including genes that allow selective toxicity such as herpesvirus TK, bcl-xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

Therapeutic Methods

The myogenic cells identified and isolated by the present invention may be used for tissue reconstitution or regeneration in a human patient or other subject in need of such treatment. The cells are administered in a manner that permits them to engraft or migrate to the intended tissue site and reconstitute or regenerate the functionally deficient area.

In one embodiment the present invention provides for the therapeutic use of muscle cells enriched (selected for) by the methods discussed herein and by techniques known in the art that are CD45−, Mac1−, GlycophorinA−, CD31−, CD34−, ITGA7+ and CD56+; especially wherein said selected population of cells are capable of forming myogenic colonies. The enrichment methods may further comprise selecting for cells that are selected for ITGA7^{hi} and CD56^{intermediate}.

The differentiating cells may be administered in any physiologically acceptable excipient, where the cells may find an appropriate site for regeneration and differentiation. The cells may be introduced by injection, catheter, surgically, or the like. The cells may be frozen at liquid nitrogen temperatures and stored for long periods of time, being capable of use on thawing. If frozen, the cells will usually be stored in a 10% DMSO, 90% FCS or in commercially available freezing medium such as Cryostor™ (Stem Cell Technologies, Vancouver, BC, Canada). Once thawed, the cells may be expanded by use of growth factors and/or feeder cells associated with progenitor cell proliferation and differentiation, as is known by one of ordinary skill in the art.

The cells of this invention can be supplied in the form of a pharmaceutical composition, comprising an isotonic excipient prepared under sufficiently sterile conditions for human administration. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells. Additional agents may be included in the composition including, for example and without limitation, anti-inflammatory agents, pain medications, growth factors, and the like.

The subject methods are useful for both prophylactic and therapeutic purposes. Thus, as used herein, the term “treating” is used to refer to both prevention of disease, and treatment of a pre-existing condition. The treatment of ongoing disease, to stabilize, slow the progression of or improve the clinical symptoms of the patient, is a particularly important benefit provided by the present invention. Such treatment is desirably performed prior to loss of function in the affected tissues; consequently, the prophylactic therapeutic benefits provided by the invention are also important. Evidence of therapeutic effect may be any diminution in the severity of disease, e.g., a decrease in progression rate of the disease. The therapeutic effect can be measured in terms of clinical outcome or can be determined by immunological or biochemical tests.

The dosage of the therapeutic formulation will vary widely, depending upon the nature of the condition, the frequency of administration, the manner of administration, the clearance of the agent from the host, and the like. The initial dose can be larger, followed by smaller maintenance doses. The dose can be administered as infrequently as weekly or biweekly, or more often fractionated into smaller doses and administered daily, semi-weekly, or otherwise as needed to maintain an effective dosage level. One of ordinary skill in the art, with the aid of the present specification, can determine effective dose regimes without undue experimentation.

Disease Conditions

Diseases of interest for treatment with the subject cells, particularly allogeneic cells and/or genetically modified autologous cells include muscular dystrophies. Duchenne dystrophy is an X-linked recessive disorder characterized by progressive proximal muscle weakness with destruction and regeneration of muscle fibers and replacement by connective tissue. Duchenne dystrophy is caused by a mutation at the Xp21 locus, which results in the absence of dystrophin, a protein found inside the muscle cell membrane. It affects 1 in 3000 live male births. Symptoms typically start in boys aged 3 to 7 yr. Progression is steady, and limb flexion contractures and scoliosis develop. Firm pseudohypertrophy (fatty and fibrous replacement of certain enlarged muscle groups, notably the calves) develops. Most patients are confined to a wheelchair by age 10 or 12 and die of respiratory complications by age 20.

Becker muscular dystrophy is a less severe variant, also due to a mutation at the Xp21 locus. Dystrophin is reduced in quantity or in molecular weight. Patients usually remain ambulatory, and most survive into their 30s and 40s.

Among the non-dystrophic myopathies are congenital and metabolic myopathies, including glycogen storage diseases and mitochondrial myopathies. Congenital myopathies are a heterogeneous group of disorders that cause hypotonia in infancy or weakness and delayed motor milestones later in childhood. An autosomal dominant form of nemaline myopathy is linked to chromosome 1 (tropomyosin gene), and a recessive form to chromosome 2. Other forms are caused by mutations in the gene for the ryanodine receptor (the calcium release channel of the sarcoplasmic reticulum) on chromosome 19q. Skeletal abnormalities and dysmorphic features are common. Diagnosis is made by histochemical and electron microscopic examination of a muscle sample to identify specific morphologic changes.

Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external ophthalmoplegia, the Kearns-Sayre syndrome (with ophthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and fatal). Muscle biopsy specimens stained with modified Gomori's trichrome stain show ragged red fibers due to excessive accumulation of mitochondria. Biochemical defects in substrate transport and utilization, the Krebs cycle, oxidative phosphorylation, or the respiratory chain are detectable. Numerous mitochondrial DNA point mutations and deletions have been described, transmitted in a maternal, nonmendelian inheritance pattern. Mutations in nuclear-encoded mitochondrial enzymes occur.

Glycogen storage diseases of muscle are a group of rare autosomal recessive diseases characterized by abnormal accumulation of glycogen in skeletal muscle due to a specific biochemical defect in carbohydrate metabolism. These diseases can be mild or severe. In a severe form, acid maltase deficiency (Pompe's disease), in which 1,4-glucosidase is absent, is evident in the first year of life and is fatal by age 2. Glycogen accumulates in the heart, liver, muscles, and nerves. In a less severe form, this deficiency may produce proximal limb weakness and diaphragm involvement causing hypoventilation in adults. Myotonic discharges in paraspinal muscles are commonly seen on electromyogram, but myotonia does not occur clinically. Other enzyme deficiencies cause painful cramps after exercise, followed by myoglobinuria. The diagnosis is supported by an ischemic exercise test without an appropriate rise in serum lactate and is confirmed by demonstrating a specific enzyme abnormality.

Channelopathies are neuromuscular disorders with functional abnormalities due to disturbance of the membrane conduction system, resulting from mutations affecting ion channels. Myotonic disorders are characterized by abnormally slow relaxation after voluntary muscle contraction due to a muscle membrane abnormality.

Myotonic dystrophy (Steinert's disease) is an autosomal dominant multisystem disorder characterized by dystrophic muscle weakness and myotonia. The molecular defect is an expanded trinucleotide (CTG) repeat in the 3′ untranslated region of the myotonin-protein kinase gene on chromosome 19q. Symptoms can occur at any age, and the range of clinical severity is broad. Myotonia is prominent in the hand muscles, and ptosis is common even in mild cases. In severe cases, marked peripheral muscular weakness occurs, often with cataracts, premature balding, hatchet facies, cardiac arrhythmias, testicular atrophy, and endocrine abnormalities. Mental retardation is common. Severely affected persons die by their early 50s.

Myotonia congenita (Thomsen's disease) is a rare autosomal dominant myotonia that usually begins in infancy. In several families, the disorder has been linked to a region on chromosome 7 containing a skeletal muscle chloride channel gene. Painless muscle stiffness is most troublesome in the hands, legs, and eyelids and improves with exercise. Weakness is usually minimal. Muscles may become hypertrophied. Diagnosis is usually established by the characteristic physical appearance, by inability to release the handgrip rapidly, and by sustained muscle contraction after direct muscle percussion.

Familial periodic paralysis is a group of rare autosomal dominant disorders characterized by episodes of flaccid paralysis with loss of deep tendon reflexes and failure of muscle to respond to electrical stimulation. The hypokalemic form is due to genetic mutation in the dihydropyridine receptor-associated calcium channel gene on chromosome 1q. The hyperkalemic form is due to mutations in the gene on chromosome 17q that encodes a subunit of the skeletal muscle sodium channel (SCN4A).

Libraries

The cells of this invention can be used to prepare a cDNA library relatively uncontaminated with cDNA preferentially expressed in cells from other lineages. For example, myogenic precursors are collected by centrifugation at 1000 rpm for 5 min, and then mRNA is prepared from the pellet by standard techniques (Sambrook, et al., supra). After reverse transcribing into cDNA, the preparation can be subtracted with cDNA from other progenitor cells, or end-stage cells from the myocyte or any other developmental pathway. This technique is known to those of skill in the art as subtractive hybridization.

The cells of this invention can also be used to prepare antibodies that are specific for markers of myocytes and their precursors. Polyclonal antibodies can be prepared by injecting a vertebrate animal with cells of this invention in an immunogenic form. Further, the cell membranes and/or cell membrane proteins can be isolated for use in the preparation of antibodies that are specific for markers of myocytes and their precursors. Production of monoclonal antibodies is described in standard references. Specific antibody molecules can also be produced by contacting a library of immunocompetent cells or viral particles with the target antigen and growing out positively selected clones. See, Marks, et al., New Eng. J. Med. 335:730, 1996, and McGuiness, et al., Nature Biotechnol. 14:1449, 1996. A further alternative is reassembly of random DNA fragments into antibody encoding regions, as described in EP patent application no. 1,094,108 A.

The antibodies in turn can be used to identify or rescue cells of a desired phenotype from a mixed cell population, for purposes such as co-staining during immunodiagnosis using tissue samples, and isolating precursor cells from terminally differentiated myocytes and cells of other lineages.

Of particular interest is the examination of gene expression in the myogenic cells of the invention. The expressed set of genes may be compared against other subsets of cells, against other stem or progenitor cells, against adult muscle tissue, and the like, as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, RNA sequences or in Northern blots containing poly A+mRNA. One of skill in the art can readily use these methods to determine differences in the size or amount of mRNA transcripts between two samples. Subtractive hybridization can also be used to determine differences in gene expression between the isolated cells of the present invention and other cell populations.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu, et al, Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854 and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon, et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e., data deviating from a predetermined statistical distribution, and calculating the relative binding affinity of the targets from the remaining data. The resulting data can be displayed as an image with the intensity in each region varying according to the binding affinity between targets and probes.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

In another screening method, the test sample is assayed for the level of polypeptide of interest. Diagnosis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g., fluorescein, rhodamine, Texas red, etc.). The absence or presence of antibody binding can be determined by various methods, including flow cytometry of dissociated cells, microscopy, radiography, scintillation counting, etc. Any suitable alternative methods of qualitative or quantitative detection of levels or amounts of differentially expressed polypeptide can be used, for example ELISA, western blot, immunoprecipitation, radioimmunoassay, etc.

Screening Assays

The cells of the present invention are also useful for in vitro assays and screening to detect factors that are active on cells of the myocyte lineage. Of particular interest are screening assays for agents that are active on human cells. The identification of factors (i.e., agents, therapeutics, chemicals, etc.) can be performed by a wide variety of assays. These include, for example, immunoassays for protein binding; determination of cell growth, differentiation and functional activity; production of factors; and the like.

In one embodiment the present invention uses for screening assays muscle cells enriched (selected for) by the methods discussed herein and by techniques known in the art that are CD45−, Mac1−, GlycophorinA−, CD31−, CD34−, ITGA7+ and CD56+; especially wherein said selected population of cells are capable of forming myogenic colonies. The enrichment methods may further comprise selecting for cells that are selected for ITGA7^{hi} and CD56^{intermediate}.

In a particular embodiment, a composition comprising a population of human myogenic progenitor cells, said cells isolated from human muscle tissue and selected for CD45−, Mac1−, GlycophorinA−, CD31− and CD34−, ITGA7+ and CD56+ expression and a pharmacological agent are contacted with one or more pharmacological agent(s) to create treated cells and the rate of cell growth is determined as compared to an essentially identical population of control cells that were not contacted with the pharmacological agent(s), wherein an increased cell growth rate of the treated cells as compared to the control cells is indicative of promotion of cell growth by the pharmacological agent. The method may further comprise using cells that are selected for ITGA7^{hi} and CD56^{intermediate} expression.

In screening assays the subject cells, usually a culture comprising the subject cells, is contacted with the agent of interest and the effect of the agent assessed by monitoring output parameters, such as expression of markers, cell viability, and the like, as compared to suitable control conditions. The cells may be freshly isolated, cultured, genetically altered as described above, or the like. The cells may be environmentally induced variants of clonal cultures: e.g., split into independent cultures and grown under distinct conditions, for example with or without virus; in the presence or absence of other cytokines or combinations thereof. The manner in which cells respond to an agent, particularly a pharmacologic agent, including the timing of responses, is an important reflection of the physiologic state of the cell and is indicative of the agent's potential ability to be used for therapeutic purposes.

Measurable parameters are quantifiable components or metabolic products of cells, particularly components and metabolic products that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g., mRNA, DNA, etc. or a portion derived from such a cell component or combinations thereof. While most parameters will provide a quantitative readout, in some instances a semi-quantitative or qualitative result will be acceptable. Variability can be expected and a range of values for each of the set of test parameters can be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like. Candidates identified as suitable for a particular purpose may be further studied.

In addition to complex biological agents, such as viruses, candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g., ground water (preferably concentrated), sea water (preferably concentrated), mining waste, etc.; biological samples, e.g., lysates prepared from crops, botanical and animal tissue samples, etc.; manufacturing samples, e.g., time course during preparation of pharmaceuticals; as well as libraries of compounds prepared for analysis; and the like. Samples of interest include compounds being assessed for potential therapeutic value, i.e., drug candidates.

The term “samples” also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g., under nitrogen, frozen, refrigerated, desiccated, hermetically sealed, or a combination thereof. The volume of sample used is sufficient to allow for measurable detection, usually from about 0.1:1 to 1 ml of a biological sample is sufficient but depends on sample concentration and test model system used.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

Agents are screened for biological activity by adding the agent to at least one and usually a plurality of cell samples (i.e., cells in culture) or animal model systems comprising a measurable population of target sample cells (e.g., cells transplanted into a host), usually in conjunction with cells lacking the agent. The change in parameters in response to the agent is measured, and the result evaluated by comparison to reference cultures, e.g., in the presence and absence of the agent, obtained with other agents, etc.

Preferred agent formulations do not include additional components, such as preservatives, that may have a significant effect on the overall formulation. Thus preferred formulations consist essentially of a biologically active compound and a physiologically acceptable carrier or diluent, e.g., water, ethanol, DMSO, etc. However, if a compound is liquid without a solvent, the formulation may consist essentially of the compound itself.

A plurality of assays may be run in parallel with different agent concentrations to obtain a differential response to the various concentrations, i.e., a titration curve). As known in the art, determining the effective concentration of an agent typically uses a range of concentrations resulting from 1:10, or other log scale or scale (i.e., 1:3, 1:6, etc.), dilutions. The concentrations may be further refined with a second series of dilutions, if necessary. Typically, one of these concentrations serves as a negative control, i.e., at zero concentration or below the level of detection of the agent or at or below the concentration of agent that does not give a detectable change in the phenotype. A positive control may also be used to, for example, ensure cell culture viability.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g., by expressing them as green fluorescent protein chimeras inside cells (for a review see Jones, et al., (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel, et al., eds, John Wiley & Sons, New York, N.Y., 2000; Freeman, et al., (1999) Biotechniques 26(1):112-225; Kawamoto, et al., (1999) Genome Res 9(12):1305-12; and Chen, et al., (1998) Genomics 51(3):313-24, for examples. Other measurable binding agents may also be used, such as horseradish peroxidase or known radioactive labels.

The invention also provides a pharmaceutical pack or kit comprising one or more containers filled with one or more of the ingredients of the pharmaceutical compositions of the invention as well as instructions for use. Associated with such container(s) can be a notice in the form prescribed by a governmental agency regulating the manufacture, use or sale of pharmaceuticals or biological products, which notice reflects approval by the agency of manufacture, use or sale for human administration.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference. Further, each referenced journal publication or other non-patent publication is representative of what is well known in the art and available to one of ordinary skill in the art at the time of the invention and, therefore, need not be repeated herein (MPEP 2164.05(a), 8th edition, revision 7).

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXPERIMENTAL

### Example 1

### Identification of Human Skeletal Muscle Stem Cells

The primary goals of this work were to identify phenotypically the human skeletal muscle precursor cell (hSMP) population, to define any age-related changes in hSMP frequency and function, to establish transplantation protocols useful for the treatment of damaged muscle, to establish a human sarcoma model system and to test potential pharmacological targets that may reverse hSMP deficiencies in aged or injured skeletal muscle.

The goal of this Example has been to determine the cell surface marker combination that identifies human skeletal muscle precursor cells and to establish protocols for their prospective isolation from human skeletal muscle. Herein we disclose the identification of a novel population of human myofiber-associated cells (CD45−MAC1−GlycophorinA−CD31−CD34−ITGA7hiCD56intermediate) that is highly enriched for human skeletal muscle precursor cells as evidenced by PAX7 and M-cadherin positivity, absence of contamination with adipogenic cells, and highly efficient in vitro myogenic differentiation capacity.

Established procedures (U.S. Pat. No. 7,749,754 to Sherwood; Sherwood, et al., Cell, 119:543-554, 2004) for use in the isolation of myofiber-associated cells from mouse skeletal muscle have been adapted and modified to isolate a physiologically similar subset of myofiber-associated cells from fresh, non-fixed human adult and fetal muscle. The identification of murine (e.g., mouse) satellite cell subpopulations that are capable of myogenic activity is of little help in the identification and isolation of similarly responding cells in humans. This is because the markers that identify the suitable murine cell population(s) have little to no relation to the markers that may identify a similarly acting human cell population(s). The difference between species in identifying suitable distinguishing cell markers is attributed to, for example, genetic differences, differences in the host's cellular microenvironment and/or host physiological differences. Thus, as is well known in the art, the identification of markers for one species is of little if any help in the identification of cell markers for a similar cell population of a differing species.

Human adult skeletal muscle specimens were obtained from the National Development and Research Institutes (NDRI, Philadelphia, Pa.), and human fetal skeletal muscle specimens from aborted, 18-20 week gestation fetuses have been obtained from Advanced Bioscience Resources (ABR, Alameda, Calif.). Human myofiber-associated cells were isolated by two-step enzymatic digestion and gentle mechanical dissociation to first obtain bulk muscle fibers, and then to liberate the mononuclear cells associated with these myofibers, as detailed below under Experimental Procedures. Flow cytometric analysis indicated that freshly isolated human myofiber-associated cells contain a phenotypically and functionally heterogeneous population of cells.

Skeletal muscle is a complex tissue, composed of multi-nucleated myofibers and a variety of myogenic (muscle-forming) and non-myogenic cells (Wagers, A. J. and Conboy, I. M., Cellular and molecular signatures of muscle regeneration: current concepts and controversies in adult myogenesis. Cell 122 (5), 659 (2005)). Myogenic cells in skeletal muscle reside primarily within the pool of muscle satellite cells, located beneath the basal lamina of mature muscle fibers (Mauro, A., Satellite cell of skeletal muscle fibers. J Biophys Biochem Cytol 9, 493 (1961)).

Human progenitor cells were isolated from non-fixed human adult and fetal muscle tissue. Isolation strategies to purify populations of myofiber-associated (MFA) cells from freshly dissociated human skeletal muscle were developed. Extensive trial and error resulted in the identification of CD45^{neg}, MAC1^{neg}, GlycophorinA^{neg}, CD34^{neg}, ITGA7^{pos(hi)}, CD56^{pos(intermediate)} cells as being highly enriched for human skeletal muscle precursor cells (hSMPs). With regard to the ITGA7^{pos(hi)} and CD56^{pos(intermediate)} markers it is noted that the designations of “hi” and “intermediate” are subsets of positively stained cells and may be denoted as “positive” as well as “hi” and “intermediate.”

The staining for the FACS sorting is the standard: about 20 mins. on ice after a blocking step of about 20 mins. on ice, as detailed below in Experimental Procedures, below.

Antibodies used (all commercially available mouse monoclonal antibodies; other suitable antibodies are available and known to those of ordinary skill in the art. It is well within the ken of one or ordinary skill in the art to test antibodies for suitable use in the present invention without undue experimentation):
CD45, clone HI30
CD11b, clone ICRF44 (MAC1)
Glycophorin, clone HIR2
CD31, clone B-B38 (PECAM-1 [Platelet Endothelial Cell Adhesion Molecule-1] blood cell marker)
CD34, clone 581
CD56, clone B159
ITGA7, clone 3C12

Flow cytometric analysis of hMFA cells revealed differential expression of 11 candidate cell surface markers (CD45, CD11b (MAC1), GlycophorinA (GlyA), β1 Integrin, CD29, CD34, CD56, ITGA7, CD90, CD13 and CXCR4). 9.1±1.7% (mean±s.d.) of fetal hMFA cells expressed hematopoietic lineage markers (CD45, MAC1 and GlyA) and CD31, a marker of endothelial cells (Andukuri et al., 2012), while expression of CD34, CD56 and ITGA7 was detected in 55±10.8%, 40.5±7.0% and 55.9±9.2% of cells, respectively (mean±s.d.). Other markers analyzed included CD29, CD90, CD13 and CXCR4, which were expressed by 90.4±9.4% (CD29), 63.9±10.3% (CD90), 38.4±3.8% (CD13) and 63.2±10.3% (CXCR4) of cells, respectively (mean±s.d). These analyses confirmed substantial heterogeneity of cell surface marker expression by hMFA cells. We next sought to exploit this heterogeneity to fractionate hMFA cell subsets with distinct differentiation potentials.

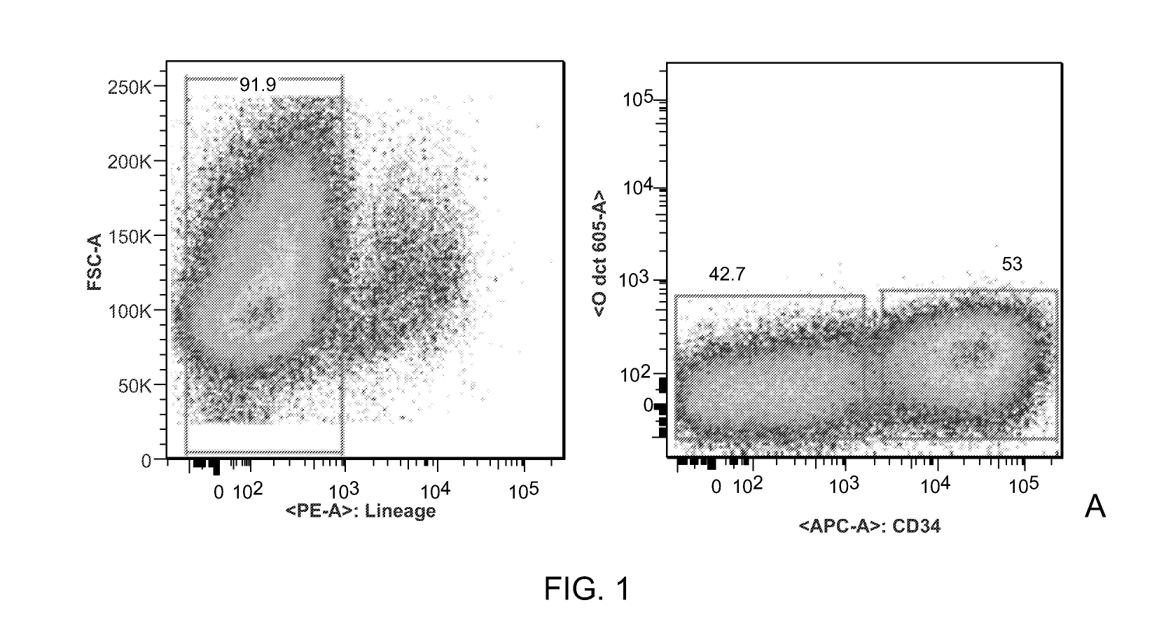

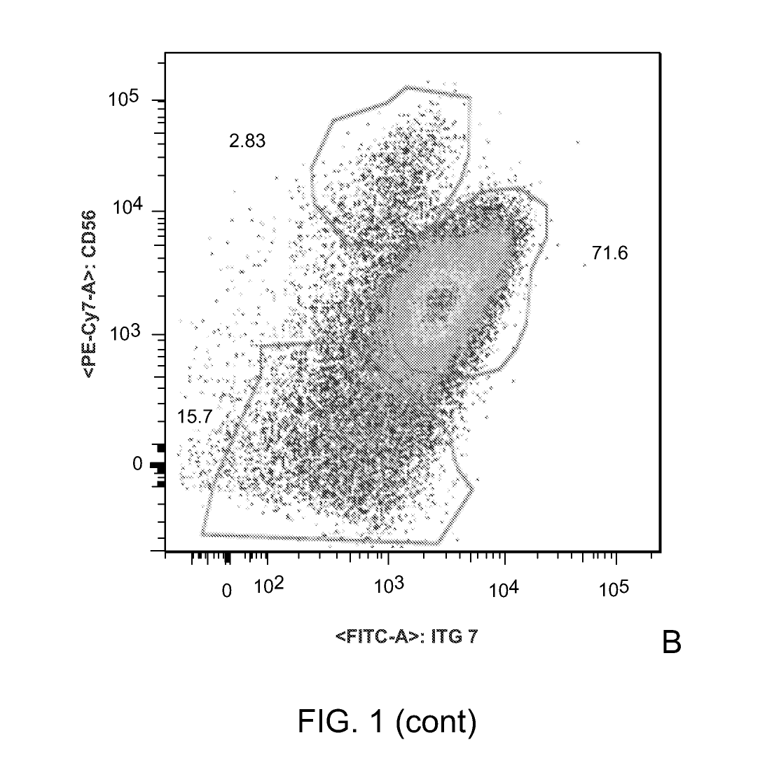

Four antibodies (anti-CD45, anti-CD11b, anti-Glycophorin and anti-CD31) were conjugated with the same fluorochrome (PE) in order to exclude everything that is positive for those markers (FIG. 1A, left panel). Cells negative for PE staining were then screened for CD34 expression. In the CD34 negative gate (FIG. 1A, right panel) the cells were separated based on the expression of CD56 and ITGA7 (FIG. 1B). Thus, this isolation profile was the result of first isolating the CD45−MAC1−Glycophorin⁻CD31−, gating on the CD34− population and then detecting CD56 and ITGA7 by immunocytochemical staining. Specific procedures are provided below in Experimental Procedures.

Satellite cells are marked by expression of the transcription factor PAX7 and function normally to support muscle maintenance and growth after birth (Seale, P., Sabourin, L. A., Girgis-Gabardo, A., Mansouri, A., Gruss, P., and Rudnicki, M. A., Pax7 is required for the specification of myogenic satellite cells. Cell 102 (6), 777 (2000)). Other mononuclear cells in skeletal muscle include non-myogenic mesenchymal cells, and infiltrating cells of the immune/inflammatory system (Sherwood, R. I., Christensen, J. L., Conboy, I. M., Conboy, M. J., Rando, T. A., and Weissman, I. L., and Wagers A. J., Isolation of adult mouse myogenic progenitors: functional heterogeneity of cells within and engrafting skeletal muscle. Cell 119 (4), 543 (2004)).

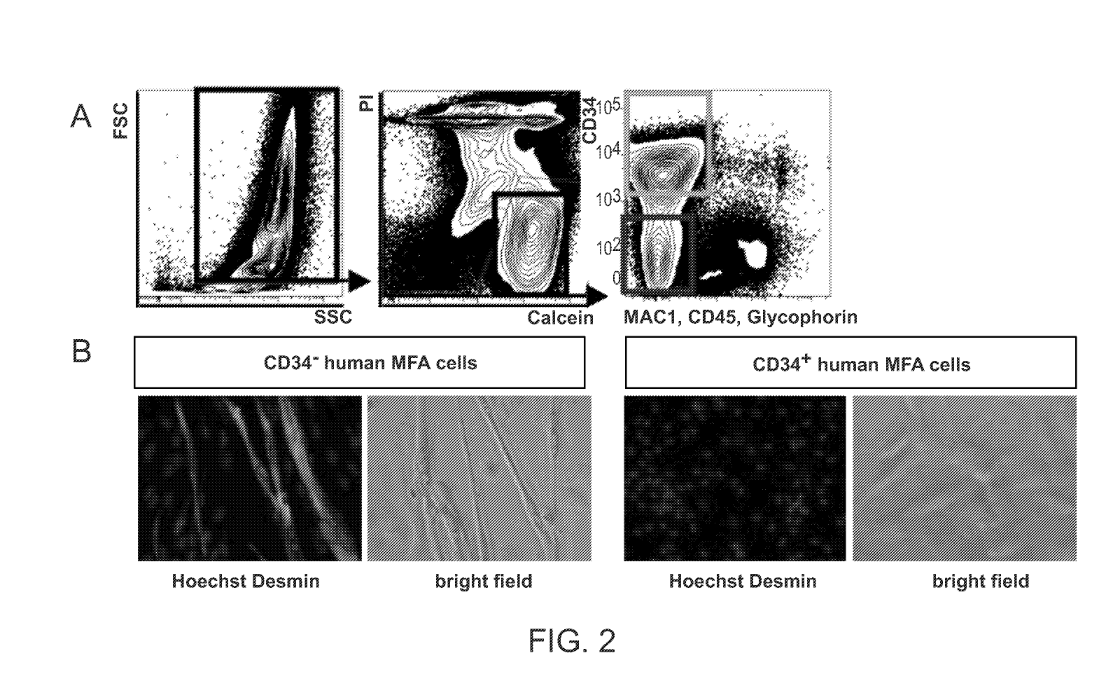

PAX7 (a transcription factor) and M-Cadherin (a cell surface antigen) represent canonical satellite cell markers. Thus, a putative human skeletal muscle precursor cell population should express nuclear PAX7 in close to 100% of cells. CD45−MAC1−GlycophorinA−CD34− cells were substantially enriched for PAX7-positive cells (32%±7% PAX7 positive), whereas CD45−MAC1−GlycophorinA−CD34+ cells were uniformly PAX7 negative (0% PAX7 positive) by immunocytochemical staining of cells freshly sorted from the pool of human myofiber-associated cells. As shown in FIG. 2A, the CD45⁻MAC1⁻Glycophorin⁻CD34⁻ subset of MFA cells (lower left-hand gate in third panel in FIG. 2A), hereafter designated “CD34⁻ cells,” was enriched for Pax7-expressing muscle satellite cells (32±9% of human CD34⁻ cells are PAX7+ as determined by immunocytochemical staining of freshly sorted cells, data not shown). CD34⁻ human MFA cells also exhibited efficient differentiation to form Desmin-positive multinucleated myotubes in vitro (FIG. 2B). In contrast, the CD45⁻MAC1⁻TER119⁻CD34⁺ subset of human MFA cells (upper left-hand gate in third panel in FIG. 2A), hereafter designated “CD34⁺ cells,” contained only PAX7-negative cells (0% PAX7+, data not shown) that completely lacked myogenic activity and adopted a fibroblastic morphology in culture (FIG. 2B).

Further sub-fractionation of the CD45−MAC1−GlycophorinA−CD34− population showed that CD45−MAC1−GlycophorinA−CD34−ITGA7hiCD56intermediate cells contained (1) 79%±5% PAX7 positive cells by imunocytochemical staining (3 independent experiments) and (2) 68.8%±7.4% M-Cadherin positive cells by flow cytometry. Thus, human CD45−MAC1−GlycophorinA−CD31−CD34−ITGA7hiCD56intermediate myofiber-associated cells are highly enriched for cells expressing human satellite cell markers, as expected for a population of human skeletal muscle precursor cells (hSMPs). As indicated elsewhere in this specification, the designations of hi and intermediate are subcategories of positive (pos or +) stained cells.

By definition, pure/highly enriched hSMPs should be highly myogenic in vitro. CD45−MAC1−GlycophorinA−CD34− cells from human muscle reliably gave rise to Desmin-positive multinucleated myofibers (replicated in >3 independent experiments performed using cells isolated from adult or from fetal human muscle specimens). CD45−MAC1−GlycophorinA−CD34+ cells, on the other hand, never gave rise to Desmin-positive, multinucleated myocytes. Recent experiments showed that the subpopulation of CD45−MAC1−GlycophorinA−CD31−CD34−ITGA7hiCD56intermediate myofiber-associated cells exhibited extremely efficient myogenic differentiation capacity as evidenced by the formation of Desmin-positive myoblasts and multinucleated yotubes in 3 independent experiments using cells isolated from human fetal muscle. This was consistent with the data, outlined above, indicating that CD45−MAC1−GlycophorinA−CD31−CD34−ITGA7^{hi}CD56^{intermediate} myofiber-associated cells are highly enriched for Pax7+, M-cadherin+hSMPs.

In addition to muscle-forming cells, mammalian skeletal muscle contains non-myogenic cells (Sherwood R I, et al. (2004) Isolation of adult mouse myogenic progenitors: functional heterogeneity of cells within and engrafting skeletal muscle. Cell 119(4):543-554; Joe A W, et al. (2010) Muscle injury activates resident fibro/adipogenic progenitors that facilitate myogenesis. Nat Cell Biol 12(2):153-163; Schulz T J, et al. (2011) Identification of inducible brown adipocyte progenitors residing in skeletal muscle and white fat. Proc Natl Acad Sci USA 108(1):143-148; Uezumi A, Fukada S, Yamamoto N, Takeda S, & Tsuchida K (2010) Mesenchymal progenitors distinct from satellite cells contribute to ectopic fat cell formation in skeletal muscle. Nat Cell Biol 12(2):143-152), including adipogenic precursors, fibroblastic and hematopoietic lineage cells. A population of putative skeletal muscle precursor cells should be selectively myogenic and should not differentiate into cells of other mesodermal lineages, such as adipocytes. We recently established conditions to induce adipogenic differentiation of human myofiber-associated cells and showed that human CD45−MAC1−GlycophorinA−CD34+ myofiber-associated cells are adipogenic. Here it was shown that CD45−MAC1−GlycophorinA−CD31−CD34−ITGA7hiCD56intermediate myofiber-associated cells were incapable of adipogenic differentiation in vitro. These observations further support the conclusion that CD45−MAC1−GlycophorinA−CD31−CD34−ITGA7hiCD56intermediate myofiber-associated cells are highly enriched for human skeletal muscle precursor cells, and indicate that this population of sorted cells is not contaminated with adipogenic cells.

Thus, we have identified and isolated distinct human skeletal muscle cell populations of tissue-specific precursor cells that can be purified on the basis of the unique combinations of cell surface markers identified in this specification. Significantly, each of these cell populations represents a potential target for malignant transformation.

### Example 2

### Regeneration of Human Muscle Tissue

Mouse skeletal muscle precursor cells represent bona fide tissue stem cells, capable of in vivo engraftment and reseeding of the mouse satellite cell pool post injury. By definition, a human putative skeletal muscle precursor cell population should meet the same criteria. In this Example, we injected distinct subpopulations of freshly sorted human myofiber-associated cells, including CD45−MAC1−GlycophorinA−CD34− cells and CD45−MAC1−lycophorinA−CD31−CD34−ITGA7hiCD56intermediate cells into the cardiotoxin pre-injured tibialis anterior muscle of NSG (NOD.SCID IL2Rγ−/−) mice, Recipient muscles were harvested 3-4 weeks post injection. Engraftment of human cells was detected by species-specific staining for human Spectrin and human Lamin A/C according to protocols previously established in the lab and known to one of ordinary skill in the art.

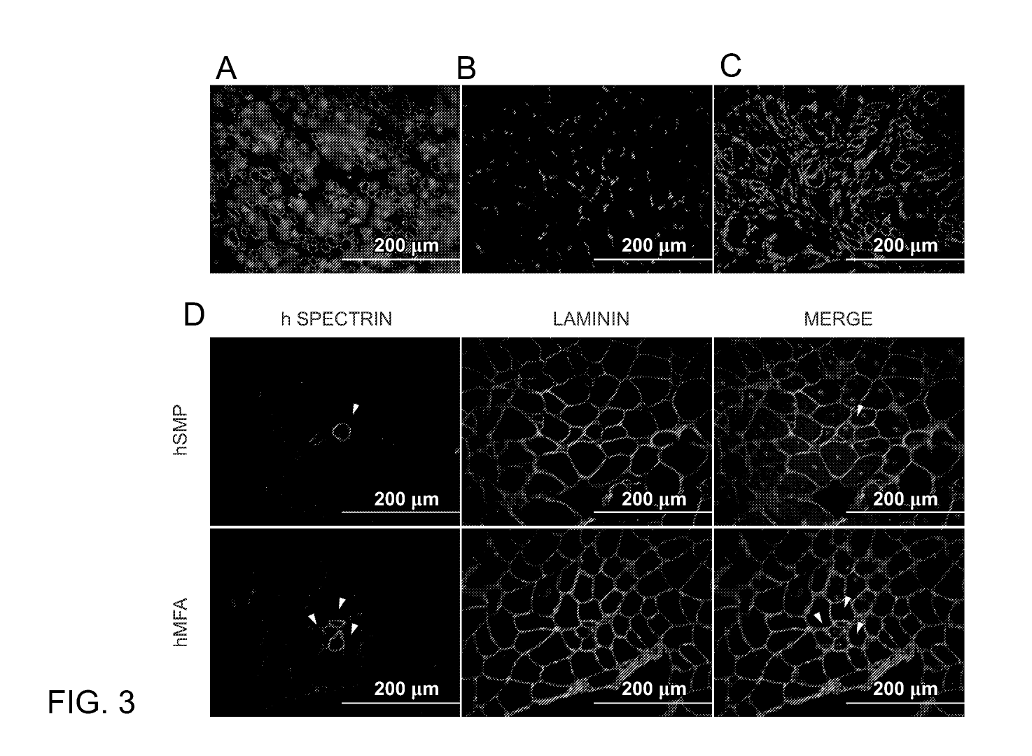

To determine the ability of sorted hMFA cells to contribute to muscle regeneration in vivo, we adapted previously published protocols to detect engraftment of unfractionated human myogenic cells in mouse skeletal muscle (Ehrhardt, et al., 2007, Disord, 17:631-638). Freshly isolated cells were injected directly into the cardiotoxin pre-injured tibialis anterior muscles of immunodeficient NSG recipient mice, transplanted muscles were harvested 3 weeks after transplantation, and engraftment of human cells was detected by staining with human species-specific antibodies against the human membrane protein Spectrin (h-Spectrin). Species-specific staining for h-Spectrin was strongly positive in human muscle sections and uniformly absent in mouse muscle sections as shown in FIGS. 3A-B. Engraftment of unfractionated hMFA cells as evidenced by the presence of h-Spectrin-positive cells on serial sections of transplanted muscles was determined in 2 independent experiments using 2 donors (human engraftment detected in 4 out of 4 transplanted mice (2 of 2 human donors), 40,000-100,000 cells transplanted per mouse, FIG. 3C). Notably, we observed substantial variability in the number of engrafted, h-Spectrin-positive cells in mice transplanted with cells obtained from different donors (FIG. 3C and FIG. 3D, lower left panel). Engraftment of human cells to form myofibers was confirmed by co-staining for h-Spectrin and Laminin, a membrane protein expressed in both mouse and human myofibers. Co-alignment of h-Spectrin and Laminin positive cells on serial sections of transplanted muscles confirmed that engrafted hMFA cells contributed to the architecture of recipient muscles (FIG. 3D, lower panels).

To determine the in vivo myogenic activity of sorted CD34⁻CD56^{int}ITGA7^{hi} hSMPs, such cells were isolated from 10 individual donors and transplanted into the cardiotoxin pre-injured limb muscles of 22 recipient mice in 10 independent experiments (40,000-100,000 hSMPs transplanted per mouse). Again, although marked variability in engraftment efficiencies was observed between donors, engraftment of human cells was detected via species-specific staining for h-Spectrin in 6 out of 22 mice transplanted (4 out of 10 human donors) (FIG. 3D, upper left panel). Again, alignment of h-Spectrin and Laminin positive cells on serial sections of transplanted muscles confirmed contribution to new myofibers from engrafted CD34⁻CD56^{int}ITGA7^{hi}fetal hSMPs. Thus, hSMPs exhibit myogenic engraftment ability in vivo, suggesting that these cells can serve as a viable source of muscle regenerative progenitors.

### Example 3

### The Transcriptional Signatures of Fetal hMFA Cell Subsets are Consistent with Lineage-Specific Differences in their Differentiation Capacitates

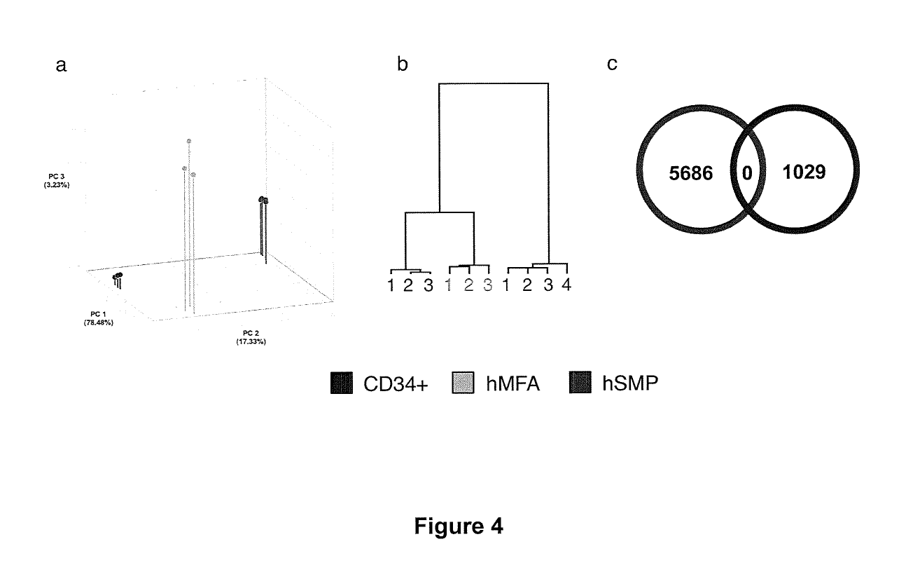

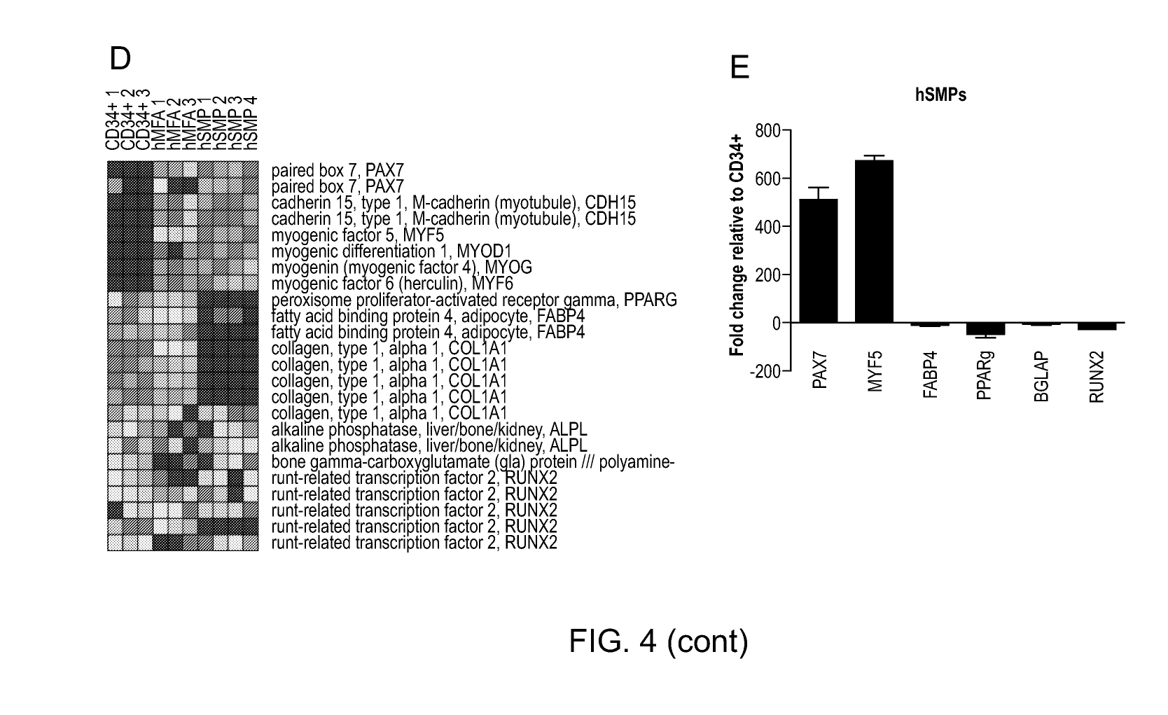

To gain deeper insights into the molecular underpinnings of CD34⁻CD56^{int}ITGA7^{hi} hSMPs and CD34⁺ adipogenic precursors within the hMFA cell pool, the transcriptional profile of these functionally distinct cell populations, as compared to unfractionated hMFA cells, was evaluated using the 0133 plus 2 Affymetrix microarray platform. Principal component analysis (PCA, FIG. 4A) and hierarchical cluster analysis (FIG. 4B) showed clustering of fetal hSMPs, CD34⁺ cells and hMFAs into 3 transcriptionally distinct cell populations. Comparison of hSMPs to hMFAs identified 5686 differentially regulated probesets, and comparison of CD34⁺ cells to hMFAs yielded 1029 differentially regulated probesets (>1.5-fold difference up or down and p<0.05). Notably, there was no overlap between these groups of differentially regulated genes (FIG. 4C). Ingenuity pathway analysis (Ingenuity Systems, Inc., Redwood City, Calif.) revealed that within the group of genes most highly upregulated in hSMPs as compared to CD34⁺ cells (>5-fold difference, p<0.01, total 346 genes), the 25 top-scoring functions involved muscle development, differentiation or function (Table 1). Interestingly, within the group of genes most highly upregulated in CD34⁺ cells versus hSMPs (>5-fold difference, p<0.01, total 854 genes), the 7 top-scoring functions involved solid tumor malignancy (Table 2).

TABLE 1

| Function Annotation | p-value | # Molecules |
|---|---|---|
| congenital myopathy | 2.27E−13 | 12 |
| development of muscle | 1.55E−12 | 28 |
| muscle contraction | 8.88E−10 | 19 |
| myogenesis | 1.91E−09 | 15 |
| development of striated muscle | 2.64E−09 | 15 |
| centronuclear myopathy | 4.17E−09 | 8 |
| morphogenesis of muscle | 2.15E−08 | 7 |
| morphogenesis of cardiac muscle | 2.28E−08 | 6 |
| myopathy | 4.54E−08 | 17 |
| contraction of striated muscle | 7.60E−08 | 10 |
| development of skeletal muscle | 2.03E−07 | 11 |
| quantity of skeletal muscle cells | 4.70E−07 | 6 |
| size of skeletal muscle cells | 5.38E−07 | 8 |
| quantity of muscle cells | 2.70E−06 | 10 |
| differentiation of myoblasts | 5.59E−06 | 9 |
| cardiomyopathy | 8.83E−06 | 14 |
| hypertrophic cardiomyopathy | 1.62E−05 | 6 |
| differentiation of muscle cells | 1.92E−05 | 14 |
| muscular dystrophy | 2.10E−05 | 9 |
| congenital anomaly of musculoskeletal system | 2.19E−05 | 21 |
| dyskinesia | 2.97E−05 | 28 |
| formation of thin filaments | 3.83E−05 | 3 |
| contraction of skeletal muscle | 3.90E−05 | 4 |
| dilated cardiomyopathy | 4.20E−05 | 9 |
| diameter of muscle cells | 4.75E−05 | 5 |

TABLE 2

| Function Annotation | p-value | # Molecules |
|---|---|---|
| solid tumor | 3.78E−29 | 227 |
| epithelial tumor | 5.88E−29 | 230 |
| carcinoma | 1.32E−28 | 222 |
| metastatic colorectal cancer | 3.82E−28 | 49 |
| metastasis | 1.32E−25 | 85 |
| adenocarcinoma | 4.52E−25 | 110 |
| Cancer | 1.09E−24 | 275 |

Gene expression by hSMPs and CD34+ cells of certain myogenic lineage (PAX7, MYF5, M-CADHERIN/CDH15, MYOD, MYOG), adipogenic lineage (PPARG, FABP4, COL1A1) and osteogenic lineage (ALPL, BGLAP, RUNX2) was also specifically analyzed in the microarray dataset (FIG. 4D). Expression of myogenic genes was upregulated in hSMPs, whereas adipogenic genes were upregulated in CD34⁺ cells (FIG. 4D). Expression of osteogenesis-associated genes was detected in both hSMPs and CD34⁺ cells (FIG. 4D), consistent with the osteogenic activity of both cell populations in vitro. Finally, we confirmed differential expression of lineage-specific genes (PPARG, FABP4, BGLAP, RUNX2, PAX7 and MYF5) in fetal hSMPs and CD34⁺ cells by qRT-PCR (FIG. 4E). PPARG, FABP4, BGLAP and RUNX2 transcript levels were reduced in hSMPs compared to CD34⁺ cells. In contrast, PAX7 and MYF5 transcript levels were markedly increased in hSMPs, consistent with their myogenic precursor function. Thus, sorted hSMPs and CD34⁺ cells possess transcriptional signatures that are highly consistent with their distinct differentiation potentials.

### Example 4

### Adult Muscle Shows Reduced Content hSMPs

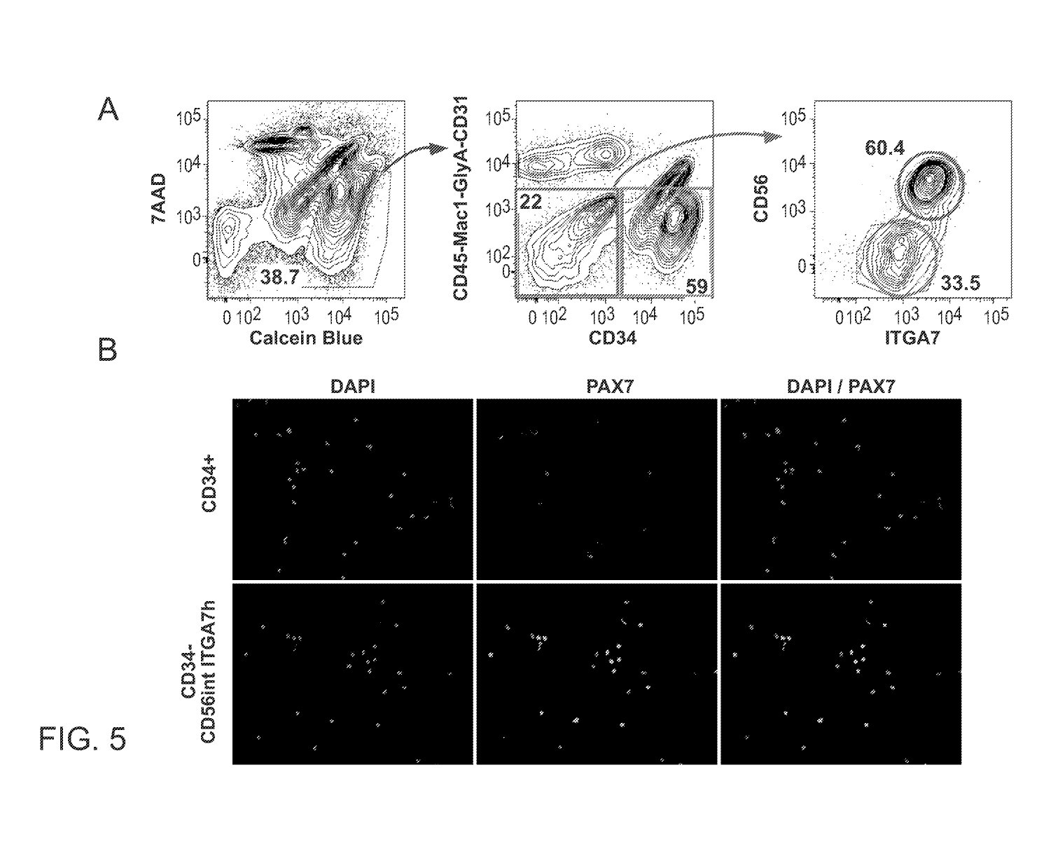

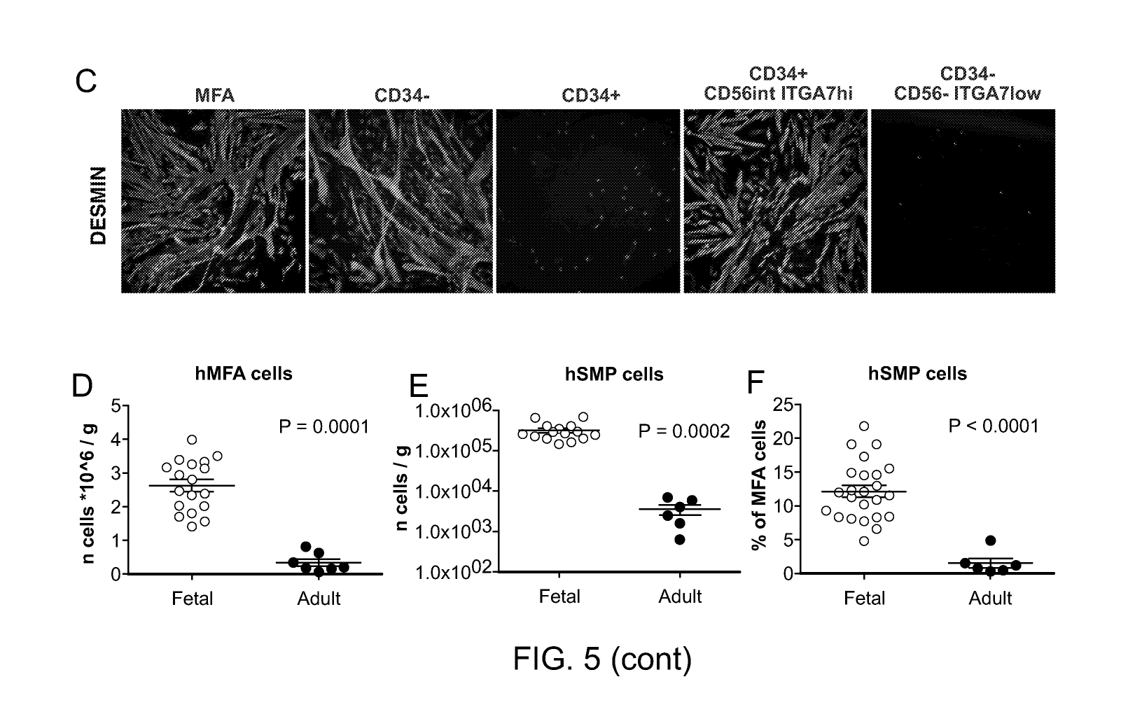

Satellite cells are established in skeletal muscle during fetal development and maintained postnatally to support muscle regenerative activity throughout life. To determine whether the cell surface marker combination identified herein as marking hSMPs in human fetal muscle would similarly mark muscle satellite cells in adult tissue, differential expression of hSMP markers (FIG. 5A), PAX7 enrichment (FIG. 5B) and myogenic differentiation capacity (FIG. 5C) in hMFA cells obtained from discarded human adult muscle was evaluated.

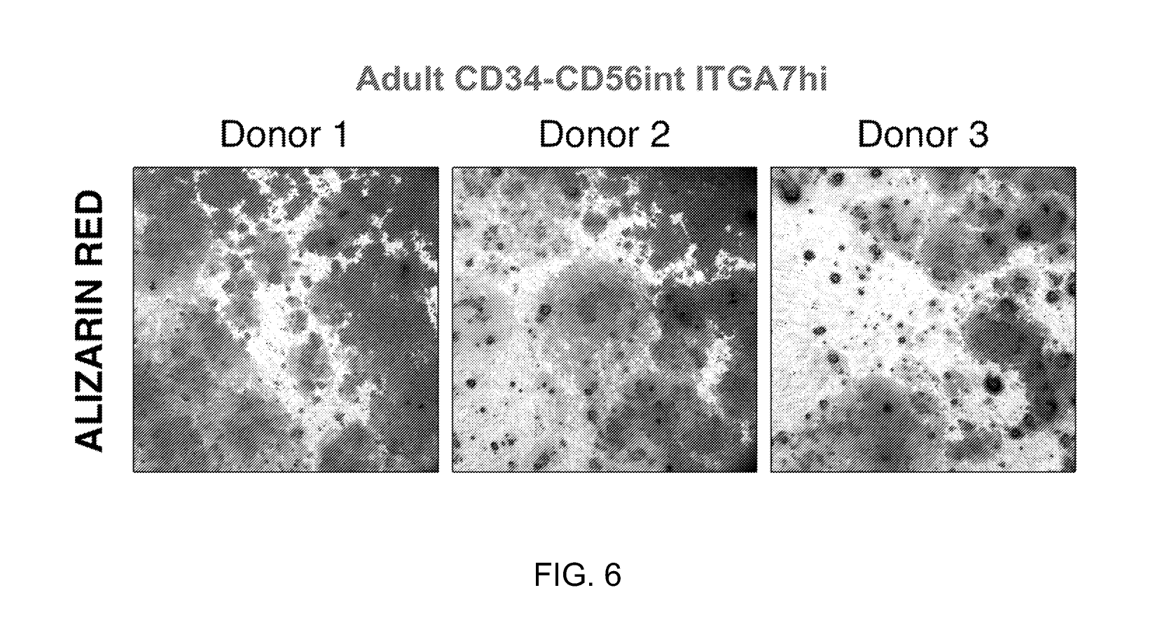

FACS analysis indicated clear separation of CD34⁺ and CD34⁻ cell subsets within the pool of viable CD45⁻CD11b⁻GlyA⁻CD31⁻ adult hMFA cells (FIG. 5A). As in fetal muscle, all myogenic differentiation capacity was contained within the CD34⁻ subset of adult human MFA cells, whereas CD34⁺ cells were uniformly non-myogenic (FIG. 5C). However, within the pool of CD34⁻ adult hMFA cells, expression of CD56 and ITGA7 discriminated only two cell populations—CD34⁻CD56⁻ITGA7^{low} cells and CD34⁻ CD56^{int} ITGA7^{hi} cells. The CD34⁻CD56^{hi}ITGA7^{low} cell population detected in fetal muscle was not present in adult muscle (FIG. 5A). We confirmed selective enrichment of PAX7-expressing cells (89±7%, mean±s.d.; FIG. 5B) and of myogenic activity (FIG. 5C, second panel from right) in adult CD34⁻CD56^{int}ITGA7^{hi} hMFA cells. Finally, analogous to fetal hSMP cells, adult CD34⁻CD56^{int}ITGA7^{hi} cells exhibited osteogenic differentiation activity (FIG. 6), in addition to their myogenic function. These data indicate that the cell surface phenotype of PAX7+ human myogenic precursors (CD45⁻CD11b⁻GlyA⁻CD31⁻CD34⁻CD56^{int}ITGA7^{hi}) is maintained from fetal life to adulthood. Notably, however, the total number of these myogenic cells differed drastically in fetal versus adult muscle. hMFA cell numbers were significantly lower in adult muscle (mean of 0.4×10⁶ (adult) or 2.5×10⁶ (fetal) hMFA cells per gram of muscle tissue; p=0.0001, FIG. 5D), and the percent of hSMPs among MFA cells was also lower (mean of 12.2±1.7% (fetal) vs 1.5±1.7% (adult); p<0.0001, FIG. 5E). This translated into an ˜2 log reduction in the total number of hSMPs in adult as compared to fetal skeletal muscle (mean 3.3×10⁵ (fetal) vs. 3.6×10³ (adult) hSMP cells per gram of skeletal muscle tissue; p=0.0002, FIG. 5F). Decreasing skeletal muscle precursor cell frequency with age in human skeletal muscle is consistent with previously published findings in mouse skeletal muscle (Conboy, et al., 2003, Science, 302:1575-7577).

### Example 5

### Novel Chimeric Model of Human Soft-Tissue Sarcoma in Mouse Skeletal Muscle

To dissect the cellular origins of sarcomas in skeletal muscle, a new mouse model based on ex vivo ectopic expression of Kras(G12V) and inactivation of the CDKN2A gene locus in distinct populations of freshly sorted human myofiber-associated cells was developed by the inventors. Activating mutations in Ras proteins and disruption of the CDKN2A locus, which encodes the p16/p14^{ink4A} and p19^{ARF} tumor suppressors that act upstream of Rb1 and Tp53, have previously been associated with sarcomas in skeletal muscle and, thus, represent relevant oncogenetic lesions in this tissue compartment.

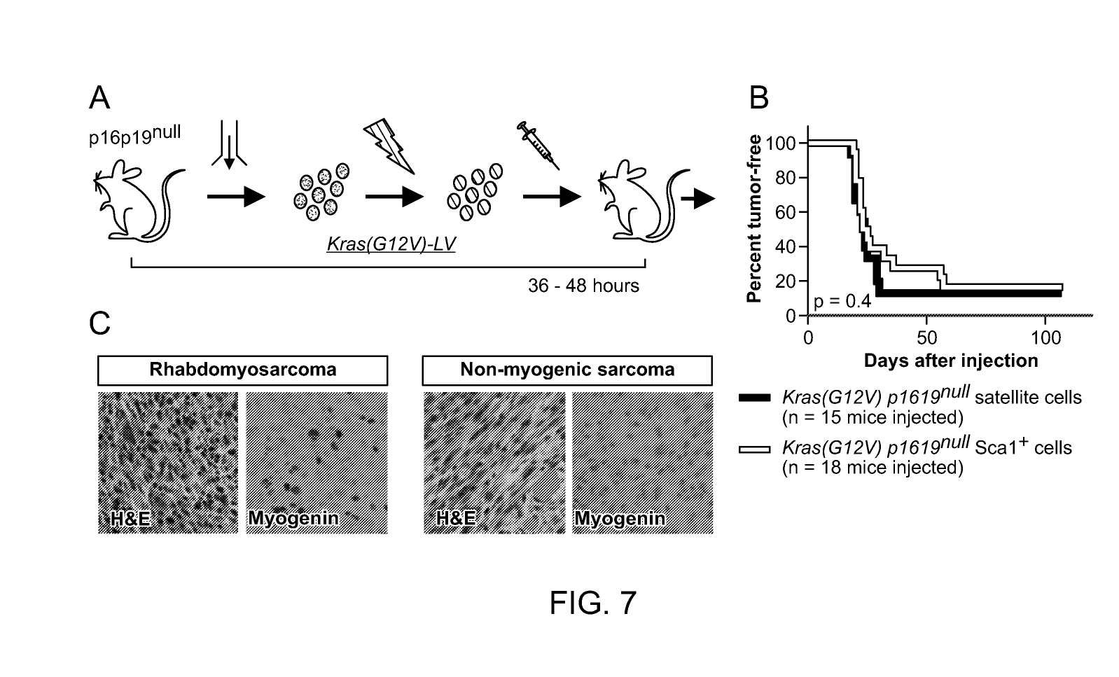

In a mouse model system utilizing isolated murine (e.g., mouse) satellite cells, a sarcoma model system was developed. To induce sarcomas, mouse satellite cells (CD45⁻MAC1⁻TER119⁻Sca1⁻β1-integrin⁺CXCR⁴⁺: Sca1− cells) or mouse Sca1⁺ cells (CD45⁻MAC1⁻TER119⁻Sca1⁺: Sca1+ cells) (see, U.S. Pat. No. 7,749,754 to Sherwood, et al.) were freshly isolated (using the FACS-based strategy discussed in Experimental Procedures with suitable antibodies) from p16p19^{null} mice, infected with Kras(G12V)-pGIPZ-IRES-GFP lentivirus, and injected into the cardiotoxin pre-injured gastrocnemius muscles of NOD.SCID mice (FIG. 7A). In this system, Kras(G12V)-infected p16p19^{null} muscle satellite cells give rise to pleomorphic sarcomas exhibiting myogenic features (tumor latency 18-30 days, frequency of Kras-infected p16p19^{null} satellite cells able to initiate tumors: 1 in 149, 95% confidence interval: 1/69-1/326) (FIG. 7B). FIG. 7C shows that satellite cells gave rise to pleomorphic rhabdomyosarcomas expressing Myogenin (the staining shown in the second panel of C; compare to the lack of staining in the forth panel of FIG. 7C), as well as MyoD and Desmin (not shown). Sca1+ cells induced sarcomas lacking these myogenic markers (FIG. 7C and data not shown). Similarly, to induce human sarcomas, human satellite cells will be freshly isolated (using the FACS-based strategy discussed above), infected with Kras(G12V)-pGIPZ-IRES-GFP lentivirus, and injected into the cardiotoxin pre-injured (or non-pre-injured) gastrocnemius muscles of NOD.SCID.IL2Rg−/− mice. The expectation is that similar results will be observed as with the murine satellite cells.

Experimental Kras Sarcoma Model Shares a Ras-Predominated Gene Expression Signature that is Also Enriched in Human Sarcomas.

Despite originating in discrete cells-of-origin and exhibiting profound differences in myogenic marker status, our transcriptional profiling and bioinformatic analyses of the modeled Kras; sarcomas indicate that these tumors share a common gene expression profile (data not shown). Intriguingly, we found that a subset of genes upregulated in modeled Kras sarcomas (compared to normal mouse skeletal muscle), also is upregulated in mouse models of sarcoma and in human pediatric rhabdomyosarcomas and non-rhabdomyosarcoma soft tissue sarcomas (Hettmer, S., Liu, J., Miller, C. M., Bronson, R. T., Langenau, D. M., and Wagers, A. J., Cellular context determines sarcoma phenotype in a mouse model of soft-tissue sarcoma, PNAS, U.S.A., 2011, 108(50):20002-20007). These data led us to identify a subset of 146 sarcoma-associated genes (represented by 194 Affymetrix probes) that are concordantly upregulated in mouse modeled Kras sarcomas and human soft-tissue sarcomas. This gene-set contains a substantial fraction (˜4/5) of genes that likewise are induced in Ras-infected cell lines, suggesting that Ras activation may be of broad significance to sarcoma biology (Hettmer, S., Liu, J., Miller, C. M., Bronson, R. T., Langenau, D. M., and Wagers, A. J., Cellular context determines sarcoma phenotype in a mouse model of soft-tissue sarcoma, PNAS, U.S.A., 2011, 108(50):20002-20007). More importantly, however, these observations confirm the relevance of the Kras sarcoma model of the present invention to genetic events that occur normally in human soft-tissue sarcomas and further identify a relatively small group of novel sarcoma-associated genes that hold significant promise as new targets for therapeutic intervention.

Several candidate pathways and genes within our sarcoma-enriched gene-set previously have been linked to the clinical and biological behavior of human sarcomas. These include the cytoskeletal protein ezrin, the TGFβ-signaling pathway (linked to cell growth and myogenic differentiation in rhabdomyosarcoma (Wang, S., Guo, L., Dong, L., Li, S., Zhang, J. and Sun, M., TGF-beta1 signal pathway may contribute to rhabdomyosarcoma development by inhibiting differentiation. Cancer Sci 101 (5), 1108 (2010)), the transcription factor SOX9 (associated with human soft tissue malignancies: Cajaiba, M. M., Luo, J., Goodman, M. A., Fuhrer, K. A., and Rao, U. N., Sox9 Expression Is Not Limited to Chondroid Neoplasms: Variable Occurrence in Other Soft Tissue and Bone Tumors With Frequent Expression by Synovial Sarcomas. Int J Surg Pathol (2010)), the metalloproteinase inhibitor TIMP1 (reduced in the serum of patients with high grade soft-tissue sarcomas, and functioning normally to suppress the invasive potential of fibrosarcoma cells: Benassi, M. S., Magagnoli, G., Ponticelli, F., Pazzaglia, L., Zanella, L., Gamberi, G., Ragazzini, P., Ferrari, C., Mercuri, M. and Picci P., Tissue and serum loss of metalloproteinase inhibitors in high grade soft tissue sarcomas. Histol Histopathol 18 (4), 1035 (2003); Tanaka, K., Iwamoto, Y., Ito, Y., Ishibashi, T., Nakabeppu, Y., Sekiguchi, M. and Sugioka, Y., Cyclic AMP-regulated synthesis of the tissue inhibitors of metalloproteinases suppresses the invasive potential of the human fibrosarcoma cell line HT1080. Cancer Res 55 (13), 2927 (1995)), the cell surface proteoglycan Syndecan-1 (expressed in several human sarcoma sub-types, and able to enhance tumor growth rate and lung metastases: Orosz, Z. and Kopper, L., Syndecan-1 expression in different soft tissue tumours. Anticancer Res 21 (1B), 733 (2001); Peterfia, B., Hollosi, P., Szilak, L., Timar, F., Paku, S., Jeney, A. and Kovalszky, I., [Role of syndecan-1 proteoglycan in the invasiveness of HT-1080 fibrosarcoma]. Magy Onkol 50 (2), 115 (2006)), cyclin-dependent kinase 6 (which promotes proliferation in rhabdomyosarcoma-derived cells: Saab, R., Bills, J. L., Miceli, A. P., Anderson, C. M., Khoury, J. D., Fry, D. W., Navid, F., Houghton, P. J., and Skapek, S. X., Pharmacologic inhibition of cyclin-dependent kinase 4/6 activity arrests proliferation in myoblasts and rhabdomyosarcoma-derived cells. Mol Cancer Ther 5 (5), 1299 (2006)) and the runt-related transcription factor RUNX1 (the most frequent target of chromosomal re-arrangements in human leukemia and implicated in normal muscle homeostasis: Lichtinger, M., Hoogenkamp, M., Krysinska, H., Ingram, R., and Bonifer, C., Chromatin regulation by RUNX1. Blood Cells Mol Dis 44 (4), 287 (2010); Wang, X., Blagden, C., Fan, J., Nowak, S. J., Taniuchi, Littman, D. R., and Burden, S. J., Runx1 prevents wasting, myofibrillar disorganization, and autophagy of skeletal muscle. Genes Dev 19 (14), 1715 (2005)). These candidate sarcoma target genes were validated by qRT-PCR and immunostaining. Our immunohistochemical studies reveal complete absence of SOX9 (encoding Sex determining region Y-box 9 transcription factor) in normal human skeletal muscle (n=7) and smooth muscle (n=5), but indicate strong expression in 8 out of 24 rhabdomyosarcomas (33%), 5 out of 27 leiomyosarcomas (19%), and 1 out of 30 leiomyomas (3%) (data not shown). Thus, SOX9 reactivity is substantially more frequent among rhabdomyosarcomas and leiomyosarcomas than in benign leiomyomas and normal muscle. These data further support the relevance of our Kras; p16p19^{null} model and gene set to human sarcomas, and confirm our ability to assay expression of these target genes in human tissues.

In summary, this Kras sarcoma model will result in rapid and reproducible murine and human tumor formation in mice, and demonstrates that lineally distinct subsets of cells (all residing within the same anatomical compartment) give rise to sarcomas that are distinguishable by the presence or absence of myogenic differentiation features. These differences in tumor phenotype correlate with their origination in distinct cell lineages within skeletal muscle, highlighting the effect of the differentiation state of the cell-of-origin on the outcome of sarcoma-associated genetic lesions.

### Example 6

### Screening of Candidate Targets by In Vitro Growth Assays Using Sarcoma Cell Lines

To begin to evaluate the effects of candidate target genes in sarcoma cells, we have established in vitro assays in which the proliferation of sarcoma cell lines exposed to selected chemical compounds is measured by MTT (3-(4,5-Dimethylthiazol-2-yl)-2,5-diphenyltretrazolium bromide) assay at defined time points. All experiments are carried out in low-passage cell lines established from modeled Kras rhabdomyosarcomas of satellite cell origin. For comparison, we also studied the effects of these compounds in the human embryonal rhabdomyosarcoma cell line RD. In these studies, we focused first on a small set of readily available chemical compounds (obtained from D. Guertin and L. Rubin at the University of Massachusetts and the Harvard Stem Cell Institute, respectively), whose targets include the TGFβ and mammalian target of rapamycin (mTOR) signaling pathways. As discussed above, the TGFβ pathway was implicated in sarcoma pathogenesis by our prior bioinformatics analyses. mTOR was tested as well because Ras pathway activation has been reported to promote mTOR signaling (Carriere, A., Romeo, Y., Acosta-Jaquez, H. A., Moreau, J., Bonneil, E., Thibault, P. Fingar, D. C., Roux, P. P., ERK1/2 Phosphorylate Raptor to Promote Ras-dependent Activation of mTOR Complex 1 (mTORC1). J Biol Chem 286 (1), 567 (2011)), and many genes on our list appear to interact with this signaling pathway (data not shown). Finally, gene ontology analyses of the 146 sarcoma-associated genes discussed above identified 61 genes that function in cell growth and proliferation (i.e., key cellular activities often regulated by mTOR).

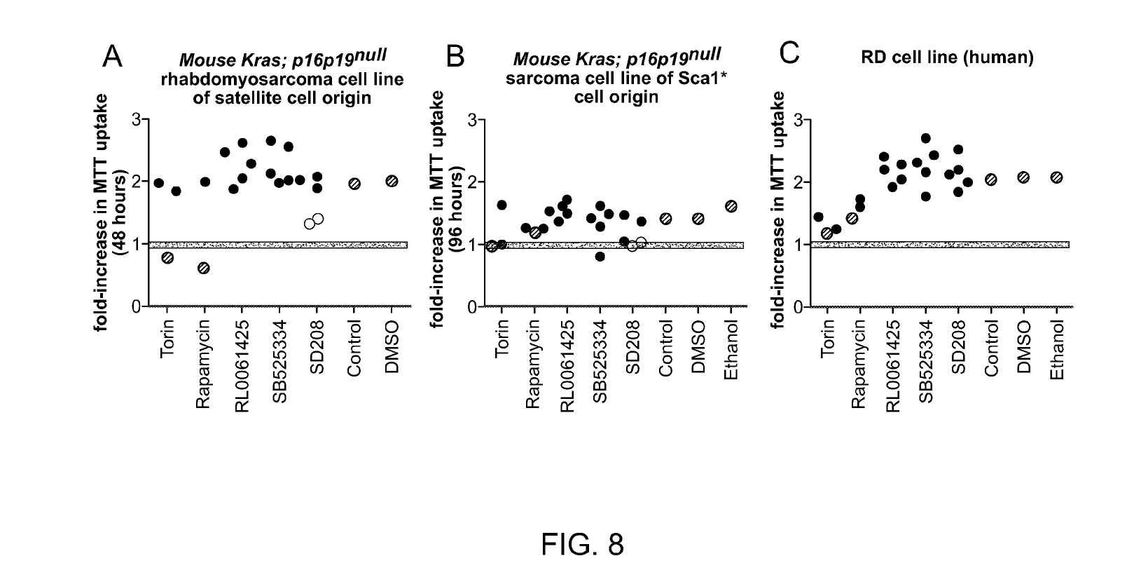

Cell lines from the modeled sarcomas were exposed to graded concentrations of mTOR inhibitors (Rapamycin, Torin) or TGF inhibitors (RL0061425, SB525334, SD208). In vitro screening of candidate targets. Sarcoma cells were exposed to increasing concentrations of Torin (10, 50, 250 nMol), Rapamycin (10, 50, 100 nMol), RL0061425 (10, 50, 100, 500, 1000 nMol), SB525334 (1, 10, 50, 100, 500 nMol), SD208 (10, 50, 100, 500, 1000 nMol), and Ethanol and DMSO (as a control). Cell growth was evaluated by determining the fold-increase in MTT-uptake over a defined time period (48 hours for the fast-growing mouse Kras; p16p1^{9null} rhabdomyosarcoma cell line and 96 hours for the slower-growing mouse Kras; p16p1^{9null} sarcoma and RD lines). mTOR inhibitors (Torin at 250 nMol and Rapamycin at 100 nMol concentration) reduced the growth of all three cell lines. The TGF inhibitor SD208 inhibited the growth of the mouse sarcoma cell lines at ≧500 nMol, while the TGF inhibitors RL0061425 and SB525334 had no major effect. These data demonstrate the feasibility of small molecule screening using primary (low passage) sarcoma lines. See, FIGS. 8A, 8B and 8C.

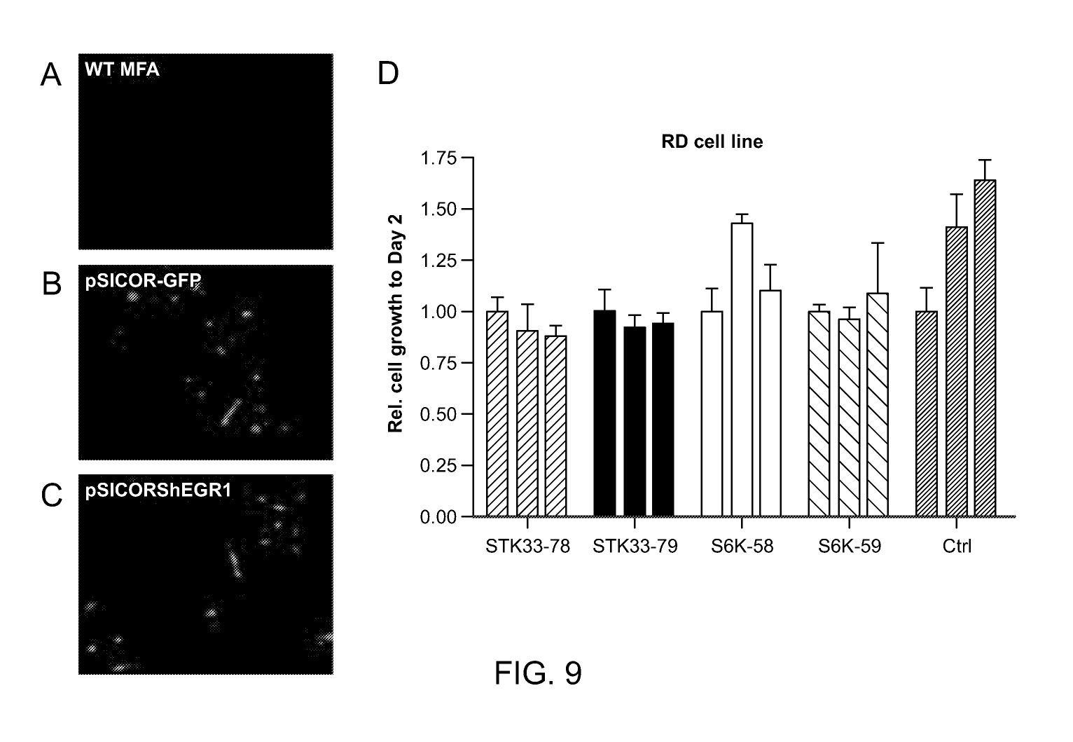

Finally, as many of the candidate target genes identified in our preliminary analyses may not be amenable to direct modulation by chemical compounds, we also have validated an alternative screening approach based on transduction of sarcoma cells with lentiviruses that carry small hairpin RNAs (shRNAs) designed to “knock-down” or “knock-out” target gene expression. As illustrated in FIG. 8, sorted mouse precursor cells (in this case satellite cells) were readily transduced with viral vectors carrying target-specific shRNAs, and knock-down of the target mRNAs had a profound influence on cell proliferation and differentiation (FIGS. 9A, 9B and 9C and data not shown). Notably, we also have successfully infected human RD rhabdomyosarcoma cells with pLKO lentiviral vectors (purchased from OpenBiosystems, Lafayette, Colo.) and found that shRNA-mediated knockdown of candidate genes (STK33 and S6K) by this approach can directly impact the proliferative activity of infected cells (FIG. 9D). These experiments confirm that shRNA-mediated knock-down of genes can be used to screen candidate genes in primary cells and in sarcoma cell lines. Results for the sarcoma model system described above using the human isolated and modified hSMPS of the present invention should be similar.

In summary, the extensive studies detailed herein have established a highly tractable model of soft-tissue sarcoma, which results in rapid and easily reproducible formation of rhabdomyosarcomas or non-myogenic sarcomas and directly recapitulates genetic events in human soft-tissue sarcomas. Furthermore, we have identified a small group of sarcoma-associated candidate genes that show broad and selective enrichment in a variety of mouse and human soft tissue sarcomas. Building upon this solid framework, we have utilized our mouse sarcoma models in sensitive chemical and genetic screening approaches. In particular, we have completed an arrayed screen at 5-fold coverage using lentiviral vectors carrying short hairpin RNAs (shRNAs) against each of the candidate anti-sarcoma genes identified by analysis of the sarcoma cells described in this invention (data not shown). This highly systematic approach provides a functional evaluation of the identified candidates and distinguishes the highest priority candidates (i.e. those whose knock-down by multiple independent shRNAs yields the most dramatic reduction in sarcoma cell proliferation) as potential anti-sarcoma agents. To further validate these new anti-sarcoma targets, we will perform analogous knock-down and chemical inhibition studies in human sarcoma cells, generated as described in this invention. We will further evaluate the impact of reduced target gene expression on tumor growth in vivo, using a mouse xenotransplantation model. These studies will identify new drug targets for use in combating rhabdomyosarcoma and non-myogenic sarcoma growth.

Additional Experimental Procedures

Muscle Dissociation and FACS sorting. Myofiber-associated and muscle interstitial cells were prepared from human tissues essentially as described (Conboy, et al., (2003) Science 302, 1575-7; Conboy, et al., (2002) Dev Cell 3, 397-409). Muscle tissue was dissected and placed in Dulbecco's Modified Eagle's Medium (Invitrogen, Carlsbad, Calif.)+0.2% collagenase type II (Invitrogen) at 37° C. and shaken for 1 hour. Collagenase digested muscle cells were poured onto a petri dish, and the collagenase solution was aspirated and replaced with Ham's F10 Medium (Invitrogen)+20% FBS.

Muscle was triturated through a fire-polished Pasteur pipet until dissociated into myofiber fragments, and medium was transferred to a 50 mL tube. Myofiber fragments were allowed to settle at 37° C. for about 12 minutes, after which the supernatant containing interstitial cells was separated from the myofiber fragments and their associated cells. This step was repeated three times. Fragments were washed three times with PBS and then dissociated in PBS+0.05% dispase (Invitrogen)+0.0125% collagenase II at 37° C., with shaking, for 30 minutes. The reaction was terminated by addition of FBS (10% of volume), and fibers were triturated three times through a micropipet tip. The preparation was then centrifuged for 1 minute at 500 RPM and supernatant containing released myofiber-associated cells was separated from settled myofiber debris. Interstitial and myofiber-associated cells were passed through nylon mesh and centrifuged at 1200 RPM. Red blood cells were lysed from interstitial preparations during a 3 minute incubation in 0.15 M ammonium chloride, 0.01 M potassium bicarbonate solution on ice.

Antibody staining was performed for 20 minutes on ice in HANKS' Balanced Salt Solution supplemented with 2% FCS and 2 mM EDTA. Prior to FACS analysis, cells were suspended in 1 μg/ml of propidium iodide (PI) to identify and exclude dead (PI⁺) cells and Calcein Blue (4.7 μg/ml) to identify live cells. Populations were sorted using a FACSria (Becton Dickinson Immunocytometry Systems, Mountain View, Calif.), provided by the Joslin Diabetes Center FACS core Facility. Flow cytometry data was analyzed using FlowJo (Treestar, San Carlos, Calif.) analysis software.

Cell Culture. 24 hours prior to plating, plates were coated with 2% Matrigel or 1 mcg/ml rat-tail collagen and 10 μg/mL natural mouse laminin (Invitrogen). Cells were plated in growth medium in 96-well tissue culture plates. Growth medium was composed of Ham's F10+20% FBS+5 ng/mL bFGF (Invitrogen)+1% penicillin/streptomycin. FGF was replaced daily. After 5-7 days, medium was changed to fusion medium: Ham's F10+1% FBS+1% penicillin/streptomycin. Cells were kept in fusion medium for a minimum of 4 days, then medium was aspirated and cells were fixed with 4% paraformaldehyde for 10 minutes and processed for immunofluorescence.

Muscle Regeneration Assays. Muscle injury was induced by injecting an anesthetized mouse with 25 μl of a 0.03 mg/ml solution of cardiotoxin (from Naja mossambica, Sigma) directly into the TA muscles. For harvesting of myofiber-associated and muscle interstitial cells, injured muscle was dissociated (see above) two days following cardiotoxin injection. Myogenic potential was also evaluated in separate experiments in which the TS muscles of GFP^{neg} mice were injured by injection of cardiotoxin, followed 24 hours later by intramuscular delivery of purified MFA cell populations. In these experiments, muscle was harvested 4 weeks after injection and analyzed by immunostaining of frozen sections.

Immunofluorescence analysis. Immunofluorescence analysis was performed on frozen sections of tibialis anterior muscles. 8 μm frozen sections were cut at −20° C. from OCT-embedded tissues using a 5030 series microtome (Bright Instruments, Huntingdon, England). Sections were permeabilized by exposure to 0.2% Triton-X for 20 minutes. Sections were blocked using Papain-digested Fab- and Fc antibodies supplemented with 5% FCS, followed by the Avidin/Biotin blocking kit (Vector Labs). Sections were stained with primary antibody overnight at 4 degrees centigrade, with secondary antibody for 60 minutes at room temperature and with tertiary antibody for 45 minutes at room temperature. Nuclei were labeled with Hoechst Dapi. Immunofluorescent labeling was analyzed both by standard fluorescence microscopy, using a Nikon Eclipse E800 microscope, with epifluorescence powered by a super high pressure mercury lamp (Nikon, Tokyo, Japan) and by laser scanning confocal microscopy, using the LSM 510 confocal Laser Scanning microscope (Zeiss, Thornwood, N.Y.) with a Coherent Mira 900 tunable Ti; Sapphire laser for 2 photon excitation, and analyzed with LSM 510 software (Zeiss), provided by the Stanford University Cell Sciences Imaging Facility. For standard epifluorescence, sequential images were acquired using a SPOT RT CCD camera (Diagnostic Instruments, Sterling Heights, Mich.) for Dapi and Alexa594, using UV-2A, HYQ Texas Red, and HYQ FITC (Nikon) filters, respectively, and electronically merged using SPOT RT software (Diagnostic Instruments). For confocal microcopy, images of serial optical sections were recorded every 1.0 μm per vertical step, and analyzed with LSM 510 and Axiovision Viewer software analysis tools (Zeiss). In all cases, appropriate negative and isotype controls demonstrated antigen-specific labeling by each of these antibodies.

Myogenic Differentiation Assay. Human MFA cell subpopulations were sorted at 1×10³ cells/well in 96 well plates, coated with 2% Matrigel (BD). Cells were expanded for 7 days in myogenic growth medium composed of Ham's F10+20% fetal bovine serum (FBS)+1% penicillin/streptomycin+25 ng/ml bFGF (Sigma)+10 ng/ml IGF-1 (Sigma). bFGF and IGF1 were replaced daily. After 7 days, growth medium was replaced with myogenic differentiation medium composed of Ham's F10+2% FBS+1% Pen-Strep. Cells were cultured in myogenic differentiation medium for 4-5 days, fixed in 4% PFA for 20 min at RT and blocked prior to staining in PBS containing 20% normal goat serum. Cells were stained with primary antibody (monoclonal mouse anti-Desmin antibody, clone D33, M0760, titer 1:50, Dako, Carpinteria, Calif.) at 4° C. overnight and with secondary antibody (goat anti-mouse Alex Fluor 594 conjugate, Invitrogen, titer 1:200) for one hour at RT. Nuclei were stained with Hoechst (2 μg/ml for 20 minutes at RT). Immunofluorescent labeling was analyzed by standard fluorescence microscopy using an Olympus IX51 microscope at 20×.

Adipogenic Differentiation Assay. Human MFA cell subpopulations were sorted at 4×10³ cells/well in 96 well plates. Cells were expanded in adipogenic growth medium composed of 60% DMEM low glucose+40% MCDB201 medium+2% FBS+1% Pen-Strep+1 nM Dexamethasone (Sigma)+0.1 mM L-Ascorbic Acid 2-Phosphate (Sigma)+ITS mix (1 in 100, Sigma)+Linoleic Acid-Albumin (1 in 100, Sigma)+25 ng/ml bFGF (Sigma) until cells reached 100% confluence (13-14 days). bFGF was replaced daily. Medium was then replaced with adipogenic induction medium composed of 60% DMEM low glucose+40% MCDB201 medium+2% FBS+1% Pen-Strep+1 μM Dexamethasone (Sigma)+5 μg/ml Insulin (Roche, Basel, Switzerland)+0.5 mM IBMX (Sigma)+1 nM T3 (Sigma)+1 μM Roziglitazone (Sigma) for 3 days. After 3 days, medium was replaced with adipogenic differentiation medium consisting of 60% DMEM low glucose+40% MCDB201 Media+2% FBS+1% Pen-Strep, 5 μg/ml Insulin (Sigma)+1 nM T3 (Sigma)+1 μM Roziglitazone (Sigma) for 4 days. Cells were fixed with 4% PFA for 20 minutes at RT, stained with Oil Red O (Sigma) for 1 hour at room temperature and then washed with dH₂O several times until the supernatant was clear. Oil Red O staining of lipid droplets within adipocytes was analyzed by standard microscopy using an Olympus IX51 inverted microscope at 20×.

Osteogenic Differentiation Assay. Human MFA cell subpopulations were sorted at 4×10³ cells/well (1×10³ cells/well in case of adult human MFA cells) in 96 well plates. Cells were expanded in Preadipocyte Medium (PM-1, ZenBio, Research Triangle Park, N.C.)+25 ng/ml bFGF (Sigma) until they reached 100% confluence (13-14 days). bFGF (Sigma) was replaced daily. Medium was then replaced with Osteoblast Differentiation Medium (OB-1, ZenBio). Cells were kept in OB-1 for 14 days, fixed in ice cold 70% ethanol for 1 hour at 4° C., stained with 2% Alizarin Red (Sigma), pH 4.2 for 10 minutes at RT and then washed with dH₂O several times until the supernatant was clear. Alizarin red staining was analyzed by standard microscopy using an Olympus IX51 microscope at 20×.

Clonal Cell Culture. Human MFA cell subpopulations were sorted at 1 cell/well in 96 well plates, coated with 1 □g/ml rat-tail collagen (Sigma) and 10 μg/ml natural mouse laminin (Invitrogen). Cells were cultured in myogenic growth medium. 25 ng/ml bFGF (Sigma) and 10 ng/ml IGF-1 (Sigma) were added daily. After 9-10 days, cell growth was evaluated by standard microscopy using an Olympus IX51 microscope at 20×. The number of wells with visible cell growth out of all wells that received one cell was determined. Cells were kept in myogenic growth conditions until they reached 100% confluence. Cells were then passaged by aspiration of medium and re-plated into 2% Matrigel (BD) coated 96-well plates. Cells were used for either myogenic or osteogenic differentiation assays as outlined above.

Transplantation studies. NOD/SCID/IL2γ^{-/-} (NSG) mice were obtained from Jackson Lab (Bar Harbor, Me.). Mice were bred and maintained at Joslin Diabetes Center. All animal experiments were approved by the Joslin Diabetes Center Institutional Animal Care and Use Committee.

The tibialis anterior (TA) muscle of anesthesized 6-8 week-old male and female NSG transplant recipients was conditioned 24 hours prior to transplantation of human cells by a single injection with 25 μl (0.03 mg/ml) of Naja mossambica mossambica cardiotoxin (CTX, Sigma). Unfractionated fetal hMFA and hSMPs cells were FACS sorted into Eppendorf tubes containing SM, spun down and re-suspended in 25 μl of SM prior to injection into the conditioned TA muscles of anesthesized recipient mice. 3 weeks after transplantation, recipient muscles were harvested, fixed by freezing in Methylbutane (Sigma) and stored at −80° C.

Engraftment was evaluated by IF staining for h-Spectrin (hum species-specific) and Laminin (muscle specific). Serial 7 μm sections of recipient TA muscles were obtained using a Cryostat. Tissue was permeabilized by exposure to Triton-X 0.2% for 20 min at room temperature and incubated 1 hour at room temperature in blocking solution (Papain digested RAM antibodies supplemented with goat Fc antibodies at 5 μg/ml and 5% FBS according to previously published protocols,¹⁶). Endogenous biotin was blocked using the Avidin/Biotin blocking kit (Vector). Tissue was stained with primary antibody (1:50, mouse anti-human SPECTRIN antibody, clone RBC2/3D5, Leica) at 4° C. overnight. Tissue was then exposed to a biotinylated goat anti-mouse secondary antibody (1 in 350, Dako) for one hour at room temperature and to Alexa-Flour-594-labeled streptavidin (1:200, Invitrogen) for 45 min at room temperature. Slides were coverslipped using Vectashield mounting media with DAPI to stain nuclei. Immunofluorescent staining was analyzed by standard fluorescence microscopy using an Olympus BX60 upright microscope at 40×.

Microarray Analysis. Unfractionated human MFA cells, hSMPs and CD34⁺hMFAs were sorted by FACS from 3-4 biologically independent human fetal skeletal muscle specimens as described above. Total RNA was obtained using TRIzol extraction. RNA quantity and quality was determined by Nanodrop and Agilent 2100 Bioanalyzer evaluation (Harvard Medical School Biopolymers Facility Service). Only samples with a purity of >99% (FIG S3) and a RIN score>7 were included in the microarray analysis. RNA was labeled and hybridized to Affymetrix microarrays (Human Genome U133 Plus 2.0). Array quality was confirmed using RMA and Affymetrix command console modules. Microarray data obtained from human MFA cell subsets were deposited in the NCBI database under accession number GSE44227.

Raw data were normalized in batch against an invariant set. Differentially regulated probesets were identified using GenePattern. Hierarchical clustering was performed in GenePattern (Broad Institute). Principal Component analysis (PCA) was performed using 3D-PCA. Row- and column-normalized and log₂-transformed data were used with the default settings of minimum expression value 120 (EV>120 for any dataset) and 20% most variable using the PCA Plot module (GenePattern, Broad Institute). The first three principle components (PC1, PC2 and PC3, respectively) were used as coordinate-axes onto which samples were projected. Pathway analysis was performed within the cluster of genes upregulated in hSMPs versus CD34⁺ cells and vice versa using Ingenuity. Cell surface location of differentially regulated transcripts was screened using Ingenuity.

PCR. Total RNA was isolated by TRIzol extraction from 2 biologically independent fetal hSMP and 3 biologically independent fetal CD34⁺ hMFA samples and reverse transcribed using Superscript III First-Strand Synthesis System for RT-PCR (Invitrogen). qRT-PCR was performed using an AV7900 PCR system (Applied Biosystem) and Taqman Gene Expression Assays (Invitrogen): PAX7 (Hs00242962_m1), MYF5 (Hs00929416_g1), PPARG (Hs01115513_m1), FABP4 (Hs01086177_m1), BGLAP (Hs01587814_g1), RUNX2 (Hs00231692_m1), GAPDH (Hs02758991_g1).

Statistics. Statistical analysis was performed using two-tailed Student's t-test for unpaired data when appropriate.

What is claimed is:

1-11. (canceled)

12. A method of enrichment for a composition comprising a population of human myogenic progenitor cells, wherein at least 80% of the cells in said population are myofiber associated, CD45−, Mac-1−, GlycophorinA−, CD31−, CD34−, ITGA7+ and CD56+, the method comprising:
a. dissociating human muscle tissue to provide a population of myofiber associated cells;
b. combining reagents that specifically distinguish CD45−, Mac1−, GlycophorinA−, CD31−, CD34−, ITGA7 and CD56, respectively, within said population of myofiber associated cells; and
c. selecting for those cells that are CD45−, Mac1−, GlycophorinA−, CD31−, CD34−, ITGA7+ and CD56+; wherein said selected population of cells are capable of forming myogenic colonies.

13. The method of claim 12, where said cells are selected for ITGA7^{hi} and CD56^{intermediate} expression.

14. The method of claim 12, wherein said reagents comprise one or more of antibodies or antibody fragments capable of distinguishing CD45, Mac1, Glycophorin A, CD31, CD34, ITGA7 and CD56, respectively.

15. The method of claim 14, wherein said each of said antibodies are selected from a group consisting of polyclonal and monoclonal antibodies.

16. The method of claim 14, wherein said antibody fragments are selected from the group consisting of Fab, F(ab′)₂, light and heavy chain fragments.

17. The method of claim 14, wherein said antibodies or antibody fragments are coupled to a label.

18. The method of claim 17, wherein said labels are selected from the group consisting of magnetic beads, biotin and fluorochromes.

19. The method of claim 12, wherein said cells are selected by flow cytometry.

20. A composition comprising a population of isolated human myogenic progenitor cells, said cells isolated from human muscle tissue and selected for CD45−, Mac1−, GlycophorinA−, CD31− and CD34−, ITGA7+ and CD56+ expression.

21. The composition of claim 20, where said cells are selected for ITGA7^{hi} and CD56^{intermediate} expression.

22. The composition of claim 20, wherein said population of isolated human myogenic progenitor cells is enriched to 50%, 75%, 80%, 90%, 95% or more of the total cell population.

23. A method of regenerating muscle tissue in a patient, comprising:
a. providing; a composition comprising a population of human myogenic progenitor cells, said cells isolated from human muscle tissue and selected for CD45−, Mac1−, GlycophorinA−, CD31− and CD34−, ITGA7+ and CD56+ expression and a patient in need of muscle tissue regeneration; and
b. introducing said composition into said patient in the location where said muscle regeneration is needed, thereby promoting the regeneration of muscle tissue.

24. The method of claim 23, where said cells are selected for ITGA7^{hi} and CD56^{intermediate} expression.

25. The method of claim 23, further comprising monitoring the regeneration of muscle tissue in the patient.

26. The method of claim 23, wherein said composition is introduced into said patient one or more times.

27. The method of claim 23, wherein said population of human myogenic progenitor cells comprise 50%, 75%, 80%, 90%, 95% or more of the total cell population.
